SEQUENCE LISTING (54 sequences)

SEQ ID NO: 1 - protein, 25 aa, Homo sapiens
ALWEAQQELGKKIKVFGPGTKLIIT

SEQ ID NO: 2 - protein, 27 aa, Homo sapiens
ACDTLGMGGEYTDKLIFGKGTRVTVEP

SEQ ID NO: 3 - protein, 24 aa, Homo sapiens
ALWEVRELGKKIKVFGPGTKLIIT

SEQ ID NO: 4 - protein, 24 aa, Homo sapiens
ACDTVGGATDKLIFGKGTRVTVEP

SEQ ID NO: 5 - protein, 24 aa, Homo sapiens
ALWEPQELGKKIKVFGPGTKLIIT

SEQ ID NO: 6 - protein, 26 aa, Homo sapiens
ACDPLLGDRYTDKLIFGKGTRVTVEP

SEQ ID NO: 7 - protein, 24 aa, Homo sapiens
ALWEVQELGKKIKVFGPGTKLIIT

SEQ ID NO: 8 - protein, 27 aa, Homo sapiens
ACDNGDTRSWDTRQMFFGTGIKLFVEP

SEQ ID NO: 9 - protein, 24 aa, Homo sapiens
ALWEDQELGKKIKVFGPGTKLIIT

SEQ ID NO: 10 - protein, 23 aa, Homo sapiens
ACDPWGTLDKLIFGKGTRVTVEP

SEQ ID NO: 11 - protein, 24 aa, Homo sapiens
ALWDQQELGKKIKVFGPGTKLIIT

SEQ ID NO: 12 - protein, 26 aa, Homo sapiens
ACAAAGGSWDTRQMFFGTGIKLFVEP

SEQ ID NO: 13 - protein, 24 aa, Homo sapiens
ALWEVKELGKKIKVFGPGTKLIIT

SEQ ID NO: 14 - protein, 23 aa, Homo sapiens
ACDTVMYTDKLIFGKGTRVTVEP

SEQ ID NO: 15 - protein, 24 aa, Homo sapiens
ALWEVEELGKKIKVFGPGTKLIIT

SEQ ID NO: 16 - protein, 25 aa, Homo sapiens
ALSPLGLGDTDKLIFGKGTRVTVEP

SEQ ID NO: 17 - protein, 24 aa, Homo sapiens
ALWEFQELGKKIKVFGPGTKLIIT

SEQ ID NO: 18 - protein, 29 aa, Homo sapiens
ACDKVSRTGGSQYTDKLIFGKGTRVTVEP

SEQ ID NO: 19 - protein, 25 aa, Homo sapiens
ALWDQSQELGKKIKVFGPGTKLIIT

SEQ ID NO: 20 - protein, 32 aa, Homo sapiens
ACDTLLGDTRRSSSWDTRQMFFGTGIKLFVEP

SEQ ID NO: 21 - protein, 24 aa, Homo sapiens
ALWEVLELGKKIKVFGPGTKLIIT

SEQ ID NO: 22 - protein, 27 aa, Homo sapiens
ACDTVSTFRGGPDKLIFGKGTRVTVEP

SEQ ID NO: 23 - protein, 23 aa, Homo sapiens
ALTGQELGKKIKVFGPGTKLIIT

SEQ ID NO: 24 - protein, 29 aa, Homo sapiens
ACDKVVGGGYAADTDKLIFGKGTRVTVEP

SEQ ID NO: 25 - protein, 24 aa, Homo sapiens
ALWEVSELGKKIKVFGPGTKLIIT

SEQ ID NO: 26 - protein, 27 aa, Homo sapiens
ACDTVVVGLGLGDKLIFGKGTRVTVEP

SEQ ID NO: 27 - protein, 25 aa, Homo sapiens
ALWEANQELGKKIKVFGPGTKLIIT

SEQ ID NO: 28 - protein, 24 aa, Homo sapiens
ACDKLGDTREKLIFGKGTRVTVEP

SEQ ID NO: 29 - protein, 23 aa, Homo sapiens
ALWEVKLGKKIKVFGPGTKLIIT

SEQ ID NO: 30 - protein, 28 aa, Homo sapiens
ACAPLGDRGSWDTRQMFFGTGIKLFVEP

SEQ ID NO: 31 - protein, 24 aa, Homo sapiens
ALWEASELGKKIKVFGPGTKLIIT

SEQ ID NO: 32 - protein, 27 aa, Homo sapiens
ACEPLRTGGPKVDKLIFGKGTRVTVEP

SEQ ID NO: 33 - protein, 24 aa, Homo sapiens
ALWEAQELGKKIKVFGPGTKLIIT

SEQ ID NO: 34 - protein, 27 aa, Homo sapiens
ACDSGGYSSWDTRQMFFGTGIKLFVEP

SEQ ID NO: 35 - protein, 24 aa, Homo sapiens
ALWEVQELGKKIKVFGPGTKLIIT

SEQ ID NO: 36 - protein, 24 aa, Homo sapiens
ACDRLLGDTDKLIFGKGTRVTVEP

SEQ ID NO: 37 - protein, 25 aa, Homo sapiens
ALWEAHQELGKKIKVFGPGTKLIIT

SEQ ID NO: 38 - protein, 24 aa, Homo sapiens
ACDSLGDSVDKLIFGKGTRVTVEP

SEQ ID NO: 39 - protein, 24 aa, Homo sapiens
ALWEDLELGKKIKVFGPGTKLIIT

SEQ ID NO: 40 - protein, 26 aa, Homo sapiens
ACDTVSWGKNTDKLIFGKGTRVTVEP

SEQ ID NO: 41 - protein, 24 aa, Homo sapiens
ALWEVRELGKKIKVFGPGTKLIIT

SEQ ID NO: 42 - protein, 27 aa, Homo sapiens
ACDTIVSGYDGYDKLIFGKGTRVTVEP

SEQ ID NO: 43 - protein, 23 aa, Homo sapiens
ALWVQELGKKIKVFGPGTKLIIT

SEQ ID NO: 44 - protein, 25 aa, Homo sapiens
ACDVLGDTEADKLIFGKGTRVTVEP

SEQ ID NO: 45 - protein, 25 aa, Homo sapiens
ALWEVRQELGKKIKVFGPGTKLIIT

SEQ ID NO: 46 - protein, 27 aa, Homo sapiens
ACDTVSQRGGYSDKLIFGKGTRVTVEP

SEQ ID NO: 47 - protein, 24 aa, Homo sapiens
ALWESKELGKKIKVFGPGTKLIIT

SEQ ID NO: 48 - protein, 29 aa, Homo sapiens
ACEGLGATQSSWDTRQMFFGTGIKLFVEP

SEQ ID NO: 49 - protein, 23 aa, Homo sapiens
ALWGGELGKKIKVFGPGTKLIIT

SEQ ID NO: 50 - protein, 26 aa, Homo sapiens
ACDLLGDTRYTDKLIFGKGTRVTVEP

SEQ ID NO: 51 - protein, 27 aa, Homo sapiens
ALWDIPPGQELGKKIKVFGPGTKLIIT

SEQ ID NO: 52 - protein, 28 aa, Homo sapiens
ACDTLGETSSWDTRQMFFGTGIKLFVEP

SEQ ID NO: 53 - DNA, 9111 bp, Artificial Sequence
  Description of Artificial Sequence Synthetic polynucleotide
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagctaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatcgaattcgccaccatgctttcccttctccacgcaagtacgctcgccgttttgggcgctctttgtgtgtatggagcaggtcatcttgagcaaccgcagatttcctccaccaagactttgtccaagaccgcgcgcttggagtgcgtggtgtcaggaattaccatctcagcgaccagcgtttactggtaccgcgagcggccaggagaagtgatacaattcttggtatcaataagctacgatggaacagttcggaaagaatctggcattccatccggtaaatttgaggtcgatcggattcccgaaacttcaacctccacgctgaccatccacaatgtagagaagcaggatattgcgacgtattactgtgcgctttgggaagcacagcaggaactgggcaaaaaaataaaagtttttggaccaggaacaaaactgataattacggataaacagcttgatgcagatgtgtccccaaaacctacaattttcttgccttccatagccgagactaagctccaaaaagctggaacttatctttgcctcctggagaaattctttcctgatgtgattaagatccattgggaggagaagaaatcaaatacgattctcggcagccaagaaggcaacaccatgaaaacgaatgatacctacatgaagtttagttggctgacggtgcctgagaaatctctggacaaagagcacaggtgtattgtgaggcacgaaaacaacaaaaatggtgtggaccaggaaatcatattccccccgataaagactgatgtaattacaatggaccccaaagataattgcagcaaagacgccaatgatactttgctgcttcagctgaccaacactagcgcctactatatgtacttgcttctgttgctgaagtctgtcgtatacttcgcaatcatcacatgttgtttgctcaggaggaccgcgttttgttgcaacggtgagaaatctagagccaagcggggctctggcgagggcagaggctctctgctgacctgcggagatgtggaagaaaatcccggccctatgcaaagaatctcatccctcattcatctctcacttttttgggcaggggtaatgtctgctatcgaacttgttcctgaacaccagactgtaccggtatccattggggtcccggcaactcttcggtgctccatgaagggggaagccatcgggaattactatatcaactggtaccggaaaacccagggtaataccatgactttcatttatagagaaaaggacatatatggtcctggctttaaagacaatttccagggtgatatcgacatagctaagaaccttgcagtcttgaaaatcctggctcctagcgaacgagatgaaggcagctactattgtgcgtgtgacacgctcggaatgggaggggaatacactgacaaactcatcttcggaaagggtaccagagtgacagtagagccaaggagccaaccgcatacaaaaccttctgtttttgtgatgaagaatggaacgaatgttgcttgcttggtcaaagaattttatccaaaagatataagaataaatctcgtgagttcaaaaaagattacagaatttgatcccgccattgtgatatccccttccggtaagtataatgctgtaaaattgggtaaatatgaagacagcaacagcgtaacttgttctgtccaacatgataataaaacggttcactctaccgactttgaagtgaagactgattctacggatcatgttaaacccaaagagacggaaaatacaaagcagccgagtaaatcatgccataaacccaaggcaatcgttcacacagaaaaggtaaatatgatgagccttactgtcctgggactgagaatgctttttgctaagaccgttgcggtgaatttccttcttactgctaagctcttctttctctaatgagttaacctcgagggatcccccggggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 54 - DNA, 9087 bp, Artificial Sequence
  Description of Artificial Sequence Synthetic polynucleotide
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagctaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatcgaattcgccaccatgctttcccttctccacgcaagtacgctcgccgttttgggcgctctttgtgtgtatggagcaggtcatcttgagcaaccgcagatttcctccaccaagactttgtccaagaccgcgcgcttggagtgcgtggtgtcaggaattaccatctcagcgaccagcgtttactggtaccgcgagcggccaggagaagtgatacaattcttggtatcaataagctacgatggaacagttcggaaagaatctggcattccatccggtaaatttgaggtcgatcggattcccgaaacttcaacctccacgttaaccatccacaatgtagagaagcaggatattgcgacgtattactgtgcgctttgggaagtacgcgaactgggcaaaaaaataaaagtttttggaccaggaacaaaactgataattacggataaacagcttgatgcagatgtgtccccaaaacctacaattttcttgccttccatagccgagactaagctccaaaaagctggaacttatctttgcctcctggagaaattctttcctgatgtgattaagatccattgggaggagaagaaatcaaatacgattctcggcagccaagaaggcaacaccatgaaaacgaatgatacctacatgaagtttagttggctgacggtgcctgagaaatctctggacaaagagcacaggtgtattgtgaggcacgaaaacaacaaaaatggtgtggaccaggaaatcatattccccccgataaagactgatgtaattacaatggaccccaaagataattgcagcaaagacgccaatgatactttgctgcttcagctgaccaacactagcgcctactatatgtacttgcttctgttgctgaagtctgtcgtatacttcgcaatcatcacatgttgtttgctcaggaggaccgcgttttgttgcaacggtgagaaatctagagccaagcggggctctggcgagggcagaggctctctgctgacctgcggagatgtggaagaaaatcccggccctatgcaaagaatctcatccctcattcatctctcacttttttgggcaggggtaatgtctgctatcgaacttgttcctgaacaccagactgtaccggtatccattggggtcccggcaactcttcggtgctccatgaagggggaagccatcgggaattactatatcaactggtaccggaaaacccagggtaataccatgactttcatttatagagaaaaggacatatatggtcctggctttaaagacaatttccagggtgatatcgacatagctaagaaccttgcagtcttgaaaatcctggctcctagcgaacgagatgaaggcagctactattgtgcgtgtgacacggtagggggtgcaactgacaaactcatcttcggaaagggtaccagagtgacagtagagccaaggagccaaccgcatacaaaaccttctgtttttgtgatgaagaatggaacgaatgttgcttgcttggtcaaagaattttatccaaaagatataagaataaatctcgtgagttcaaaaaagattacagaatttgatcccgccattgtgatatccccttccggtaagtataatgctgtaaaattgggtaaatatgaagacagcaacagcgtaacttgttctgtccaacatgataataaaacggttcactctaccgactttgaagtgaagactgattctacggatcatgttaaacccaaagagacggaaaatacaaagcagccgagtaaatcatgccataaacccaaggcaatcgttcacacagaaaaggtaaatatgatgagccttactgtcctgggactgagaatgctttttgctaagaccgttgcggtgaatttccttcttactgctaagctcttctttctctaatgaggatcccccggggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

(19) United States
(12) Patent Application Publication
(10) Pub. No.: US 2022/0211757 A1
(43) Pub. Date: Jul. 7, 2022

(54) ENGINEERED GAMMA DELTA T CELLS AND METHODS OF MAKING AND USING THEREOF

(71) Applicant: The Regents of the University of California, Oakland, CA (US)

(72) Inventors: Lili Yang, Los Angeles, CA (US); Derek Lee, Los Angeles, CA (US); Yang Zhou, Los Angeles, CA (US); Zhe Li, Los Angeles, CA (US)

(73) Assignee: The Regents of the University of California, Oakland, CA (US)

(21) Appl. No.: 17/563,482

(22) Filed: Dec. 28, 2021

Related U.S. Application Data
(60) Provisional application No. 63/131,170, filed on Dec. 28, 2020.

(51) Int. Cl. A61K 35/17; C12N 5/0783; C12N 15/86; C12N 15/62; A61K 38/17

(52) U.S. Cl. CPC A61K 35/17; C12N 2501/51; C12N 15/86; C12N 15/625; A61K 38/1774; C12N 2506/45; C12N 2740/15043; C12N 2510/00; C12N 2501/125; C12N 2501/26; C12N 2501/24; C12N 2501/25; C12N 2501/145; C12N 2501/14; C12N 2501/2302; C12N 2501/2303; C12N 2501/2306; C12N 2501/2307; C12N 2501/2315; C12N 5/0636

(57) ABSTRACT

Aspects of the present disclosure relate to methods and compositions related to the preparation of immune cells, including engineered T cells comprising at least one exogenous γδ T cell receptor, for example one that is selected to target a specific disease or pathogen (e.g., cancer or COVID-19). The T cells may be produced from human hematopoietic stem/progenitor cells and are suitable for allogeneic cellular therapy because they do not induce graft-versus-host disease (GvHD) and resist host immune allorejection. Consequently, such cells are suitable for off-the-shelf use in clinical therapy.

### CROSS-REFERENCE TO RELATED APPLICATIONS

This application claims the benefit under 35 U.S.C. Section 119(e) of co-pending and commonly-assigned U.S. Provisional Patent Application Ser. No. 63/131,170, filed on Dec. 28, 2020, and entitled “ENGINEERED GAMMA DELTA (γδ) T CELLS AND METHODS OF MAKING AND USING THEREOF” which application is incorporated by reference herein.

### TECHNICAL FIELD

Embodiments of the disclosure concern at least the fields of immunology, cell biology, molecular biology, and medicine.

### BACKGROUND OF THE INVENTION

Gamma delta (γδ) T cells are a small subpopulation of T lymphocytes having the ability to bridge innate and adaptive immunity. The majority of γδ T cells in adult human blood exhibit Vγ9Vδ2 T cell receptors and respond to small phosphorylated nonpeptide antigens, called phosphoantigens (pAgs), which are commonly produced by malignant cells (see, e.g., Yang et al., Immunity 50, 1043-1053.e5 (2019)). Unlike conventional αβ T cells, γδ T cells do not recognize polymorphic classical major histocompatibility complex (MHC) molecules and are therefore free of graft versus host disease (GvHD) risk when adoptively transferred into an allogeneic host. Additionally, γδ T cells have several other unique features that make them ideal cellular carriers for developing off-the-shelf cellular therapy for cancer. These features include: 1) γδ T cells have roles in cancer immunosurveillance; 2) γδ T cells have the remarkable capacity to target tumors independent of tumor antigen- and major histocompatibility complex (MHC)-restrictions; 3) γδ T cells can employ multiple mechanisms to attack tumor cells through direct killing and adjuvant effects; and 4) γδ T cells express a surface receptor, FcγRIII (CD16), that is involved in antibody-dependent cellular cytotoxicity (ADCC) and can be potentially combined with monoclonal antibody for cancer therapy (see, e.g., Lepore et al., Front. Immunol. 9, 1-11 (2018), Harrer et al., Hum. Gene Ther. 29, 547-558 (2018) and Presti et al., Front. Immunol. 8, 1-11 (2017)). Unfortunately, however, the development of an allogeneic off-the-shelf γδ T cellular product is greatly hindered by their availability—these cells are of extremely low number and high variability in humans (˜1-5% T cells in human blood), making it very difficult to produce therapeutic numbers of γδ T cells using blood cells of allogeneic human donors (see, e.g., Silva-Santos et al., Nat. Rev. Immunol. 15, 683-691 (2015)).

The conventional method of generating γδ T cells, in particular the Vγ9Vδ2 subset, for adoptive therapy involves either in vitro or in vivo expansion of peripheral blood mononuclear cell (PBMC)-derived γδ T cells using aminobisphosphonates, such as Zoledronate (ZOL). However, this methodology generates highly variable yields of γδ T cells depending on PBMC donors; and most importantly, such a γδ T cell product will typically contain bystander αβ T cells and thereby incurring GvHD risk (see, e.g., Torikai et al., Mol. Ther. 24, 1178-1186 (2016)).

Novel methods and materials that can reliably generate a homogenous monoclonal population of γδ T cells at large quantities with a feeder-free differentiation system are pivotal to developing off-the-shelf γδ T cell therapies that are useful in the treatment of a wide variety of pathological conditions.

### SUMMARY OF THE INVENTION

The ability to manufacture a therapeutic cell population or a cell population that can be used to create a therapeutic cell population “off-the-shelf” increases the availability and usefulness of new cellular therapies. Embodiments of the invention are provided to address the need for new cellular therapies, more particularly, the need for cellular therapies that are not hampered by the challenges posed in individualizing therapy using autologous cells.

As disclosed herein, we have discovered that engineered γδ T cells can be produced through γδ TCR gene-engineering of pluripotent cells (e.g., CD34⁺ stem and progenitor cells) followed by selectively differentiating the gene-engineered cells into transgenic γδ T cells in vivo or in vitro. As discussed below, such γδ T cells can further be engineered to co-express other disease-targeting molecules (e.g., chimeric antigen receptors, “CARs”) as well as immune regulatory molecules (e.g., cytokines, receptors/ligands and the like) to modulate their performance. Significantly, embodiments of these in vitro differentiated γδ T cells can be used for allogeneic “off-the-shelf” cell therapies for treating a broad range of diseases (e.g., cancers, autoimmune diseases, infections and the like).

Embodiments of the invention include materials and methods relating to the gamma and delta chain polypeptides that are disclosed in Table 1 below. For example, embodiments of the invention include compositions of matter comprising a gamma chain polypeptide and/or a delta chain polypeptide having an amino acid sequence shown in Table 1 (SEQ ID NO: 1-SEQ ID NO: 52). Related embodiments of the invention include compositions of matter comprising polynucleotides encoding a gamma chain polypeptide and/or a delta chain polypeptide having an amino acid sequence shown in Table 1 (SEQ ID NO: 1-SEQ ID NO: 52). In certain embodiments of the invention, these polynucleotides are disposed in a vector, for example an expression vector designed to express these gamma and delta chain polypeptides in a cell. One such embodiment of the invention is a composition of matter comprising an immune cell that has been transduced with an expression vector comprising a polynucleotide encoding at least one T cell receptor gamma chain polypeptide and/or the T cell receptor delta chain polypeptide having an amino acid sequence shown in Table 1 (SEQ ID NO: 1-SEQ ID NO: 52).

Embodiments of the invention also include, for example, methods of making an engineered functional T cell modified to contain at least one exogenous nucleic acid molecule encoding a T cell receptor gamma chain polypeptide and/or a T cell receptor delta chain polypeptide (e.g., as disclosed in Table 1). Typically these methods comprise transducing a pluripotent cell (e.g. a human CD34⁺ hematopoietic stem or progenitor cell) with the at least one exogenous nucleic acid molecule encoding a T cell receptor gamma chain polypeptide and/or a T cell receptor delta chain polypeptide so that the cell transduced by the exogenous nucleic acid molecule expresses a T cell receptor comprising a gamma chain polypeptide and a delta chain polypeptide; and then differentiating the transduced human cell so as to generate the engineered functional gamma delta T cell.

The methodological embodiments of the invention can include, for example, differentiating transduced pluripotent cells in vitro. In illustrative methods, transduced CD34⁺ human hematopoietic stem or progenitor cells (HSPC) can be differentiated in vitro in the absence of feeder cells; and/or cultured in medium comprising a cytokine such as one or more of IL-3, IL-7, IL-6, SCF, EPO, TPO and FLT3L, and/or in the presence of an agent selected to facilitate nucleic acid transduction efficiency such as retronectin. In certain embodiments, the method further comprises contacting the transduced cell with an agonist antigen or other stimulatory agent. In some embodiments of the invention, the method further comprises co-culturing the transduced cells with peripheral blood mononuclear cells, antigen presenting cells, or artificial antigen presenting cells. Certain embodiments of the invention further comprise expanding the pluripotent cell transduced with the nucleic acid molecule encoding a T cell receptor gamma chain polypeptide or a T cell receptor delta chain polypeptide in vitro. Alternative methods of the invention can comprise engrafting the cell transduced with the nucleic acid molecule encoding a T cell receptor gamma chain polypeptide and a T cell receptor delta chain polypeptide into a subject to generate clonal populations of the engineered cells in vivo.

In some embodiments of the invention, the engineered T cell comprises a gene expression profile characterized as being at least one of: HLA-I-negative; HLA-II-negative; HLA-E-positive; and/or expressing a suicide gene. Optionally, the engineered T cell further comprises an exogenous T cell receptor nucleic acid molecule encoding a T cell receptor alpha chain polypeptide or a T cell receptor beta chain polypeptide; and/or an exogenous nucleic acid molecule encoding a cytokine; and/or suppressed endogenous TCRs. In certain embodiments of the invention, a T cell receptor gamma chain polypeptide and/or the T cell receptor delta chain polypeptide expressed by these engineered cells comprises an amino acid sequence shown in Table 1 (SEQ ID NO: 1-SEQ ID NO: 52).

Embodiments of the invention include engineered functional gamma delta T cells produced by the methods disclosed herein. For example, embodiments of the invention include compositions of matter comprising an engineered T cell comprising a gene expression profile characterized as: HLA-I-negative; HLA-II-negative; HLA-E-positive; expressing a suicide gene; and expressing at least one exogenous T cell receptor gamma chain polypeptide and at least one exogenous T cell receptor delta chain polypeptide. In certain embodiments, a T cell receptor gamma chain polypeptide or a T cell receptor delta chain polypeptide comprises at least one amino acid sequence shown in SEQ ID NO: 1-SEQ ID NO: 52. In some embodiments of the invention, a CD34⁺ HSPCs can be isolated from cord blood (CB) or peripheral blood. In such embodiments of the invention, CB CD34⁺ HSCs can be obtained from commercial providers (e.g., HemaCare) or from established CB banks.

As the γδ T gamma/delta cellular product is an off-the-shelf product that can be used to treat patients independent of MHC restrictions, once commercialized, this cellular product has broad applications in a variety of potentially life-saving therapies. In this context, yet another embodiment of the invention is a method of treating a subject in need of gamma delta T cells (e.g., to fight a disease such as an autoimmune disease or a cancer or an infection such as COVID-19) which comprises administering to the subject an engineered functional T cell disclosed herein.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIGS. 1A-1C. Cloning of human γδ TCR Genes. FIG. 1(A): Experimental design to clone out human γδTCR. FIG. 1(B): Fluorescence-activated cell sorting (FACS) of single human γδT cells. FIG. 1(C): Representative DNA gel image showing the human TCR γ9 and β2 chain PCR products from five sorted single γδT cells.

FIGS. 2A-2B. Schematics of the Lenti/G115 and Lenti/γδT vectors. A pMNDW lentiviral vector designated for HSC-based gene therapy was chosen to deliver the γδ TCR gene. FIG. 2(A): A Lenti/G115 vector encoding the G115 γδ TCR gene. FIG. 2(B): A Lenti/γδT vector encoding a selected γδ TCR gene. The Lenti/γδT vector encoding the LYγδ1 TCR gene (see Table 1) was used in the presented studies.

FIGS. 3A-3E. Functional characterization of a cloned γδ TCR. PBMC-T cells were transduced with the Lenti/γδT vector encoding the indicated γδ TCR chains (i.e., G115, γδ1) and analyzed for their TCR expression and functionality. FIG. 3(A): Representative FACS plots showing the expression of transgenic γδ TCRs on Lenti/γδT vector transduced PBMC-T cells.

FIG. 3(B): FACS analyses of intracellular production of IFN-γ by Lenti/γδT vector transduced PBMC-T cells post ZOL stimulation. FIG. 3(C-E): Studying tumor killing of Lenti/γδT vector transduced PBMC-T cells. FIG. 3(C): Experimental design. FIG. 3(D): In vitro tumor killing of a human melanoma cell line (A375-FG) by Lenti/γδT vector transduced PBMC-T cells. FIG. 3(E): In vitro tumor killing of a human multiple myeloma cell line (MM.1S-FG) by Lenti/γδT vector transduced PBMC-T cells. Note the parental A375 and MM.1s human tumor cell lines were engineered to express firefly luciferase and green fluorescence protein dual reporters (FG). Data are presented as the mean±SEM. ns, not significant, *P<0.05, **P<0.01, ***P<0.001, ****P<0.0001, by one-way ANOVA.

FIGS. 4A-4B. Generation of HSC-γδT cells in a BLT-γδT humanized mouse model. FIG. 4(A): Experimental design to generate HSC-γδT cells in a BLT-γδT humanized mouse model. BLT, human bone marrow-liver-thymus implanted NOD.Cg-Prkdc^{scid}Il2rg^{tm1Wjl}/SzJ (NSG) mice. BLT-γδT, human γδTCR gene-engineered BLT mice. FIG. 4(B): FACS detection of HSC-γδT cells in various tissues of BLT-γδT mice, at week 25 post-HSC transfer. BLT mice received CD34⁺ HSC with mock vector transduction were included as a control (denoted as BLT-mock).

FIGS. 5A-5B. Generation of ^{Allo}HSC-γδT Cells in an ATO Culture. FIG. 5(A): Experimental design to generate ^{Allo}HSC-γδT cells in an ATO culture. FIG. 5(B): FACS plots showing the development of ^{Allo}HSC-γδT cells at Stage 1 and expansion of differentiated ^{Allo}HSC-γδT cells at Stage 2, from PBSCs.

FIGS. 6A-6D. Generation of ^{Allo}HSC-γδT Cells in A Feeder-Free Ex Vivo Differentiation Culture. CD34⁺ HSCs isolated from G-CSF-mobilized peripheral blood (denoted as PBSCs) or cord blood (denoted as CB HSCs) were transduced with a Lenti/γδT vector encoding a human γδ TCR gene, then put into the feeder-free ex vivo cell culture to generate ^{Allo}HSC-γδT cells (FIGS. 6A and 6B). Both PBSCs and CB HSCs can effectively differentiate into and expand as transgenic ^{Allo}HSC-γδT cells (FIGS. 6C and 6D).

FIGS. 7A-7D. CMC Study—^{Allo}CAR-γδT Cells. FIG. 7(A-B): A feeder-free ex vivo differentiation culture method to generate monoclonal ^{Allo}CAR-γδT cells from PBSCs in FIG. 7(A) or cord blood (CB) HSCs in FIG. 7(B). Note the high numbers of ^{Allo}CAR-γδT cells and their derivatives that can be generated from PBSCs or CB HSCs of a single random healthy donor. FIG. 7(C-D) Development of ^{Allo}CAR-γδT cells at Stage 1 and expansion of differentiated ^{Allo}CAR-γδT cells at Stage 2, from PBSCs in FIG. 7(C) or CB HSCs in FIG. 7(D).

FIGS. 8A-8B. Pharmacology study of ^{Allo}HSC-γδT cells. FIG. 8(A): Representative FACS plots are presented, showing the analysis of phenotype (surface markers) and functionality (intracellular production of effector molecules) of ^{Allo}HSC-γδT cells. Endogenous human γδ T (PBMC-γδ T) cells and conventional αβ T (PBMC-T) cells isolated and expanded from healthy donor peripheral blood were included as controls. FIG. 8(B): Representative FACS analyses of surface NK receptor expression by ^{Allo}HSC-γδT cells. Endogenous PBMC-γδ T cells, PBMC-T, and PBMC-NK cells isolated and expanded from healthy donor peripheral blood were included as controls.

FIGS. 9A-9E. In Vitro Efficacy and MOA Study of ^{Allo}HSC-γδT Cells. FIG. 9(A): Experimental design of the in vitro tumor cell killing assay. FIG. 9(B): Tumor killing efficacy of ^{Allo}HSC-γδT cells against A375-FG tumor cells (n=3). FIG. 9(C): Tumor killing efficacy of ^{Allo}HSC-γδT cells against MM.1s-FG tumor cells (n=3). FIG. 9(D): Tumor killing efficacy of ^{Allo}HSC-γδT cells against multiple human tumor cell lines (n=3). FIG. 9(E): Human tumor cell lines tested in the study. Data are presented as the mean±SEM. ns, not significant, *P<0.05, **P<0.01, ***P<0.001, ****P<0.0001, by one-way ANOVA. E: T, effector to target ratio.

FIGS. 10A-10C. In Vivo Antitumor Efficacy and MOA Study of ^{Allo}HSC-γδT Cells in an A375-FG human melanoma xenograft NSG mouse model. FIG. 10(A): Experimental design. BLI, live animal bioluminescence imaging. FIG. 10(B): BLI images showing tumor loads in experimental mice over time. FIG. 10(C): Quantification of B (n=4). Data are presented as the mean±SEM. ns, not significant, *P<0.05, **P<0.01, ***P<0.001, ****P<0.0001, by one-way ANOVA.

FIGS. 11A-11D. In Vitro Efficacy and MOA Study of ^{Allo}BCAR-γδT Cells. FIG. 11(A): Experimental design of the in vitro tumor cell killing assay. FIG. 11(B): Tumor killing efficacy of ^{Allo}BCAR-γδT cells against A375-FG melanoma cells in the absence or presence of ZOL (n=3). FIG. 11(C): Tumor killing efficacy of ^{Allo}BCAR-γδT cells against MM.1S-FG myeloma cells in the absence or presence of ZOL. BCAR-T cells and non-CAR-engineered PBMC-T cells and ^{Allo}HSC-γδT cells were included as controls (n=3). FIG. 11(D): Diagram showing the triple-mechanisms that can be deployed by ^{Allo}BCAR-γδT cells targeting tumor cells, including CAR-mediated, γδ TCR-mediated, and NK receptor-mediated paths. Data are presented as the mean±SEM. ns, not significant, *P<0.05, **P<0.01, ****P<0.0001, by one-way ANOVA. E:T, effector to target ratio.

FIGS. 12A-12D. In Vivo Antitumor Efficacy of ^{Allo}BCAR-γδT Cells (n=8). FIG. 12(A): Experimental design. FIG. 12(B): Representative BLI images showing tumor loads in experimental mice over time. FIG. 12(C): Quantification of B. FIG. 12(D): Kaplan-Meier survival curves of experimental mice over a period of 4 months post tumor challenge (n=8). Data are presented as mean±SEM. ns, not significant; ****p<0.0001 by one-way ANOVA FIG. 12(C), or log rank (Mantel-Cox) test adjusted for multiple comparisons FIG. 12(D).

FIGS. 13A-13D. In Vivo Antitumor Efficacy Study—^{Allo}BCAR-γδT Cells in combined with ZOL treatment. FIG. 13(A): Experimental design. FIG. 13(B): BLI images showing tumor loads in experimental mice over time. FIG. 13(C): Quantification of 13B (n=3). FIG. 13(D): Quantification of tumor load at day 39 post tumor challenging (n=3). Data are presented as the mean±SEM. ns, not significant, *P<0.05, **P<0.01, ****P<0.0001, by one-way ANOVA. E:T, effector to target ratio.

FIGS. 14A-14F. CMC study and in vivo persistence of ^{Allo15}CAR-γδT cells. FIG. 14(A): A feeder-free ex vivo differentiation culture method to generate monoclonal ^{Allo15}CAR-γδT cells from cord blood (CB) HSCs. Note the high numbers of ^{Allo15}CAR-γδT cells that can be generated from CB HSCs of a single random healthy donor. FIG. 14(B): Development of ^{Allo15}CAR-γδT cells at Stage 1 and expansion of differentiated ^{Allo15}CAR-γδT cells at Stage 2, from CB HSCs. FIG. 14(C): Experimental design to study the in vivo dynamics of ^{Allo/15}BCAR-γδT cells. Note the ^{Allo/15}BCAR-γδT cells were labeled with FG dual-reporters. FIG. 14(D): BLI images showing the presence of FG-labeled ^{Allo/15}BCAR-γδT cells in experimental mice overtime. FIG. 14(E): Quantification of D (n=1-2). Data are presented as the mean±SEM.

FIGS. 15A-15F. Immunogenicity Study. FIG. 15(A): An in vitro mixed lymphocyte culture (MLC) assay for the study of GvH response. FIG. 15(B): IFN-γ production from 15A (n=3). Donor-mismatched PBMC-T and PBMC-γδ T cells were included as controls. PBMCs from 3 mismatched healthy donors were used as stimulators. N, no PBMC stimulator. FIG. 15(C): An in vitro MLC assay for the study of HvG response. FIG. 15(D): IFN-γ production from C. PBMCs from 3 mismatched healthy donors were tested as responders. Data from one representative donor were shown (n=3). FIG. 15(E-F): FACS analyses of B2M/HLA-I and HLA-II expression on the indicated stimulator cells (n=3). Data are presented as the mean±SEM. ns, not significant, *P<0.05, **P<0.01, ****P<0.0001, by one-way ANOVA.

FIG. 16. Property of human γδ T cell products generated using various methods. Representative FACS plots are presented, showing the property of human γδ T cells from human PBMC culture and from ^{Allo}HSC-γδT cell culture. Tc, conventional c T cells.

FIGS. 17A-17D. ^{Allo}HSC-γδT Cells Directly Target and Kill SARS-CoV-2 Infected Cells. FIG. 17(A): Schematic showing the engineered 293T-FG, 293T-ACE2-FG, and Calu3-FG cell lines. FIG. 17(B): FACS detection of ACE2 on 293T-FG, 293T-ACE2-FG, and Calu3-FG cells. FIG. 17(C-D): In vitro direct killing of SARS-CoV-2 infected or non-infected target cells by ^{Allo}HSC-γδT cells (n=3). Data are presented as the mean±SSEM. ns, not significant, *P<0.05, **P<0.01, ****P<0.0001, by one-way ANOVA.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention.

Gamma delta (γδ) T cells normally account for 1 to 5% of peripheral blood lymphocytes in healthy individuals. Unlike classical αβ T cells that recognize specific peptide antigens presented by major histocompatibility complex (MHC) molecules, γδ T cells can recognize generic determinants expressed by cells that have become dysregulated as a result of either malignant transformation or viral infection. Consequently, γδ-T cells have the innate ability to recognize and kill a broad spectrum of tumor cell types, in a manner that does not require the existence of conventional tumor-specific antigens.

There is a need in the art for methods and materials that can reliably generate a homogenous monoclonal population of various engineered human T cells such as engineered γδ T cells in large quantities. These technologies are pivotal to developing off-the-shelf T cell therapies. Such methods and materials can, for example, provide γδ T cells that can be used in allogeneic or autologous recipient subjects for the treatment of a variety of pathological conditions including, for example, viral infections, fungal infections, protozoal infections and cancers.

As discussed below, we have discovered that engineered γδ T cells can be produced through γδ TCR gene-engineering of pluripotent human cells such as CD 34⁺ stem and progenitor cells (e.g., HSCs, iPSCs, ESCs) followed by selectively differentiating the gene-engineered stem and progenitor cells into transgenic γδ T cells in vivo and/or in vitro. As is known in the art, hematopoietic stem or progenitor cells possess multipotentiality, enabling them to self-renew and also to produce mature blood cells, such as erythrocytes, leukocytes, platelets, and lymphocytes. CD34 is a marker of human HSC, and all colony-forming activity of human bone marrow (BM) cells is found in the CD34+ fraction. See e.g., Mata et al., Transfusion. 2019 December; 59(12):3560-3569. doi: 10.1111/trf.15597.

This discovery is unexpected because developmental path of gamma delta T cells is unique and unlike the developmental paths of other T cells such as iNKT cells and αβ T cells (see, e.g., Dolens et al., EMBO Rep. 2020 May 6; 21(5):e49006. doi: 10.15252/embr.201949006. Epub 2020 and Shissier et al., Mol. Immunol. 2019; 105: 116-130). Importantly, the in vitro differentiated γδ T cells disclosed herein can be used for allogeneic “off-the-shelf” cell therapies for treating a broad range of diseases (e.g., cancer, infection, autoimmunity, etc.). Moreover, the γδ T cells can also be engineered to co-express other disease-targeting molecules (e.g., CARs) as well as immune regulatory molecules (e.g., cytokines, receptors/ligands) to enhance their performance.

Embodiments of the invention include, for example, methods of making an engineered functional T cell modified to contain at least one exogenous nucleic acid molecule (e.g., one disposed in an expression vector such as a lentiviral vector as discussed below) encoding a T cell receptor gamma chain polypeptide and/or a T cell receptor delta chain polypeptide such as a gamma chain polypeptide and/or a delta chain polypeptide having an amino acid sequence shown in Table 1 (SEQ ID NO: 1-SEQ ID NO: 52). Typically these methods comprise transducing a pluripotent human cell such as a hematopoietic stem/progenitor cell (i.e., a pluripotent stem cell, a hematopoietic stem cell, or a hematopoietic progenitor cell) with the at least one exogenous nucleic acid molecule encoding a T cell receptor gamma chain polypeptide and/or a T cell receptor delta chain polypeptide so that the human cell transduced by the exogenous nucleic acid molecule expresses a T cell receptor comprising a gamma chain polypeptide and a delta chain polypeptide; and then differentiating the transduced human cell (e.g. a hematopoietic stem/progenitor cell) so as to generate the engineered functional gamma delta T cell. In certain methodological embodiments of the invention, the T cell receptor gamma chain polypeptide and T cell receptor delta chain polypeptide encoded by the exogenous nucleic acid are selected as ones known to form a γδ T cell receptor that has been previously observed to target cancer cells or cells infected with a virus, bacteria, fungi or protozoan. Certain methods of the invention include the steps of differentiating the transduced human cell in an in vitro culture; and then further expanding these differentiated cells in an in vitro culture. In some methodological embodiments of the invention, expanding these differentiated cells in an in vitro culture is performed under conditions selected to expand the differentiated population of transduced cells by at least 2-fold, 5-fold, 10-fold or 100-fold. In some embodiments of the invention, the engineered functional gamma delta T cell is exposed to zoledronic acid.

The methodological embodiments of the invention include differentiating the transduced pluripotent human cells (e.g., human hematopoietic stem or progenitor cells) in vitro or in vivo and then expanding this differentiated population of cells. In certain embodiments, the method further comprises contacting the transduced cell with a stimulatory agent such as an agonist antigen. In some methodological embodiments of the invention, a population of γδ T cells is made by the methods disclosed herein wherein such methods do not include a cell sorting step (e.g., FACS or magnetic bead sorting) following transduction of the nuclei acids encoding the γ and δ polypeptides into the human cells. In some embodiments of the invention, the method further comprises co-culturing the transduced cells with peripheral blood mononuclear cells, antigen presenting cells, or artificial antigen presenting cells. Typically in these methods, the transduced human cell is differentiated in vitro in the absence of feeder cells; and/or the transduced hematopoietic stem or progenitor cell is cultured in medium comprising a cytokine such as one or more of IL-3, IL-7, IL-6, SCF, MCP-4, EPO, TPO, FLT3L, and/or an agent selected to facilitate nucleic acid transduction efficiency such as retronectin. Alternative methods of the invention can comprise engrafting the cell transduced with the nucleic acid molecule encoding a T cell receptor gamma chain polypeptide or a T cell receptor delta chain polypeptide into a subject (i.e., in vivo) to generate clonal populations of the engineered cell.

In some methodological embodiments of the invention, the engineered T cell is selected to comprise a certain gene expression profile, for example one characterized as being at least one of: HLA-I-negative; HLA-II-negative; HLA-E-positive; and/or expressing a suicide gene. Typically, the engineered T cell further comprises one or more exogenous T cell receptor nucleic acid molecules encoding a T cell receptor alpha chain polypeptide and a T cell receptor beta chain polypeptide; and/or one or more exogenous nucleic acid molecules encoding a cytokine; and/or suppressed endogenous TCRs. In some embodiments of the invention disclosed herein, the T cell receptor gamma chain polypeptide and the T cell receptor delta chain polypeptide comprises an amino acid sequence shown in Table 1 below. In particular embodiments, the one or more additional nucleic acids encode one or more therapeutic gene products. Examples of therapeutic gene products include at least the following: 1. Antigen recognition molecules, e.g. a CAR (chimeric antigen receptor) and/or an αβ TCR (T cell receptor), a γδ T receptor and the like; 2. Co-stimulatory molecules, e.g. CD28, 4-1BB, 4-1BBL, CD40, CD40L, ICOS; and/or 3. Cytokines, e.g. IL-1α, IL-1β, IL-2, IL-4, IL-6, IL-7, IL-9, IL-15, IL-12, IL-17, IL-21, IL-23, IFN-γ, TNF-α, TGF-β, G-CSF, GM-CSF; 4. Transcription factors, e.g. T-bet, GATA-3, RORγt, FOXP3, and Bcl-6. Therapeutic antibodies are included, as are chimeric antigen receptors, single chain antibodies, monobodies, humanized, antibodies, bi-specific antibodies, single chain FV antibodies or combinations thereof.

Embodiments of the invention also include materials and methods relating to the gamma and delta chain polypeptides that are disclosed in Table 1 below. For example, embodiments of the invention include compositions of matter comprising a gamma chain polypeptide and/or a delta chain polypeptide having an amino acid sequence shown in Table 1 (SEQ ID NO: 1-SEQ ID NO: 52). Related embodiments of the invention include compositions of matter comprising polynucleotides encoding a gamma chain polypeptide and/or a delta chain polypeptide having an amino acid sequence shown in Table 1 (SEQ ID NO: 1-SEQ ID NO: 52). In certain embodiments of the invention, these polynucleotides are disposed in a vector, for example an expression vector designed to express these gamma and delta chain polypeptides in a cell (e.g. a mammalian cell). The compositions of the invention may contain preservatives and/or antimicrobial agents as well as pharmaceutically acceptable excipient substances as required to approximate physiological conditions, such as pH adjusting and buffering agents, tonicity adjusting agents, wetting agents, detergents and the like. For such compositions, the term “excipient” is meant to include, but is not limited to, those ingredients described in Remington: The Science and Practice of Pharmacy, Lippincott Williams & Wilkins, 21st ed. (2006).

Embodiments of the invention further include engineered functional gamma delta T cells and populations of these cell produced by the methods disclosed herein. Typically, these populations consist essentially of functional gamma delta T cells (e.g., do not include conventional αβ T cells). Embodiments of the invention include compositions of matter comprising an engineered γδ T cell or T cell population disclosed herein such as one comprising a gene expression profile characterized as: HLA-I-negative; HLA-II-negative; HLA-E-positive; expressing a suicide gene; and expressing an exogenous T cell receptor gamma chain polypeptide and an exogenous T cell receptor delta chain polypeptide. Optionally, the engineered T cell further comprises an exogenous nucleic acid molecule encoding another polypeptide such as a T cell receptor alpha chain polypeptide and/or a T cell receptor beta chain polypeptide and/or an iNKT receptor polypeptide; and/or a cytokine; and/or comprises suppressed endogenous TCRs. Embodiments of the invention also include composition of matter comprising an immune cell that has been transduced with an expression vector comprising a polynucleotide encoding at least one exogenous T cell receptor gamma chain polypeptide and/or the T cell receptor delta chain polypeptide having an amino acid sequence shown in Table 1 (SEQ ID NO: 1-SEQ ID NO: 52).

Methods of treating patients with an γδ T cell or cell population as disclosed herein are also provided. Embodiments of the invention include methods of treating a subject in need of gamma delta T cells (e.g., to fight a disease such as an autoimmune disease or a cancer or an infection such as COVID-19) which comprises administering to the subject an engineered functional gamma delta T cell disclosed herein. In this way, engineered gamma delta T cells may be used to treat patients in need of therapeutic intervention. In some therapeutic embodiments of the invention, the methods include introducing one or more additional nucleic acids into the gamma delta T cells, which may or may not have been previously frozen and thawed. This use provides one of the advantages of creating an off-the-shelf gamma delta T cell.

In certain therapeutic methods of the invention, the patient has been diagnosed with a cancer. In some embodiments, the patient has a disease or condition involving inflammation, which, in some embodiments, excludes cancer. In specific embodiments, the patient has an autoimmune disease or condition. In particular aspects, the cells or cell population is allogeneic with respect to the patient. In additional embodiments, the patient does not exhibit signs of rejection or depletion of the cells or cell population. Some therapeutic methods further include administering to the patient a stimulatory molecule (e.g., alone or loaded onto APCs) that activates γδ T cells, or a compound that initiates the suicide gene product.

Treatment of a cancer patient with the γδ T cells may result in tumor cells of the cancer patient being killed after administering the cells or cell population to the patient. Treatment of an inflammatory disease or condition may result in reducing inflammation. In other embodiments, a patient with an autoimmune disease or condition may experience an improvement in symptoms of the disease or condition or may experience other therapeutic benefits from the γδ T cells. Combination treatments with γδ T cells and standard therapeutic regimens or another immunotherapy regimen(s) may be employed.

As discussed below, the figures included herewith provide examples of a number of illustrative working embodiments of the invention as well as data obtained from such embodiments of the invention.

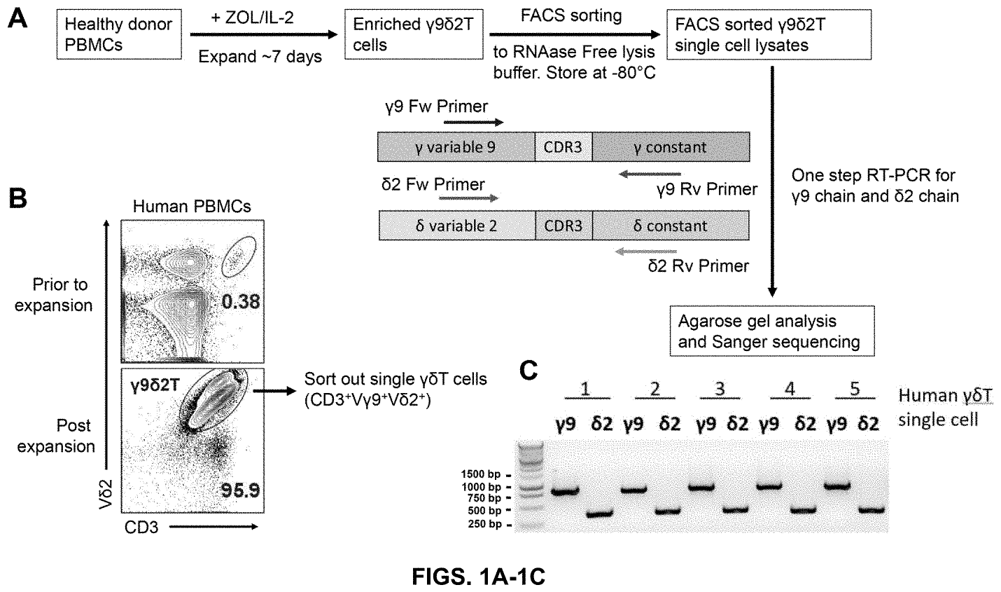

For the convenience of expression in this disclosure, we refer to a pair of γ9δ2 TCR genes as a γδTCR gene. As shown in FIG. 1, each pair of γδTCR gene contain a gamma chain and a delta chain. In some embodiments, the engineered γδ T cell comprises a nucleic acid under the control of a heterologous promoter, which means the promoter is not the same genomic promoter that controls the transcription of the nucleic acid. It is contemplated that the engineered γδ T cell comprises an exogenous nucleic acid comprising one or more coding sequences, some or all of which are under the control of a heterologous promoter in many embodiments described herein.

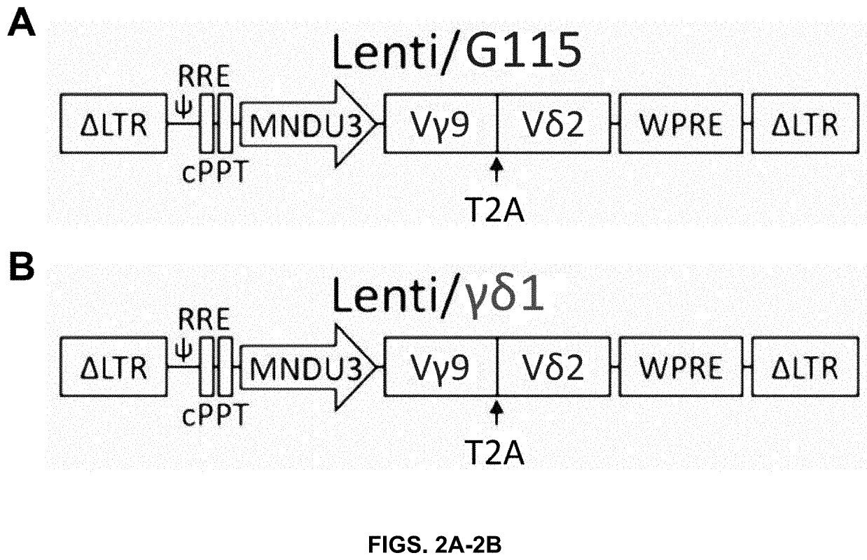

FIG. 2 shows the construction of lentiviral vectors for delivering γδ TCR genes. As shown in FIG. 2, in an illustrative embodiment of the invention, a pMNDW lentiviral vector was chosen to deliver the γδ TCR genes. This vector contains the MND retroviral LTR U2 region as an internal promoter and contains an additional truncated Woodchuck Responsive Element (WPRE) to stabilize viral mRNA, thus mediates high and stable expression of transgene in human HSCs and their progeny human immune cells. The Lenti/γδT vector was constructed by inserting into pMNDW a synthetic bicistronic gene encoding human TCRγ9-T2A-TCRδ2. Two plasmids expressing clone G115 and γδ1 from Table 1 have been constructed using this strategy (FIG. 2).

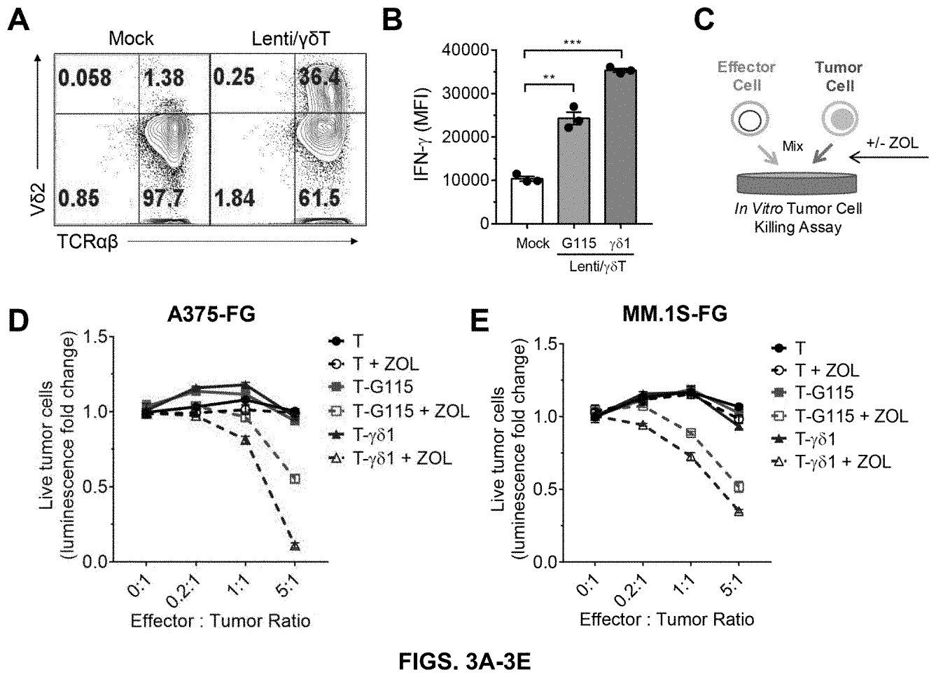

FIG. 3 shows the functional characterization of a cloned γδ TCR. As shown in FIG. 3, the gene-delivery capacity of the Lenti/γδT vector (FIG. 3A), as well as the functionality of its encoded γδTCR, were studied by transducing primary human PBMC-derived conventional αβT (denoted as PBMC-T) cells with lentivectors followed by functional tests. Notably, this lentivector mediated efficient expression of the human γδ TCR transgene in PBMC-T cells (FIG. 3B); the resulting transgenic human γδ TCRs responded to zoledronate (ZOL) stimulation, as evidenced by induced interferon (IFN)-γ production (FIG. 3C) and enhanced tumor killing when co-culturing the transduced PBMC-T cells with human tumor cells (FIGS. 3D-3F).

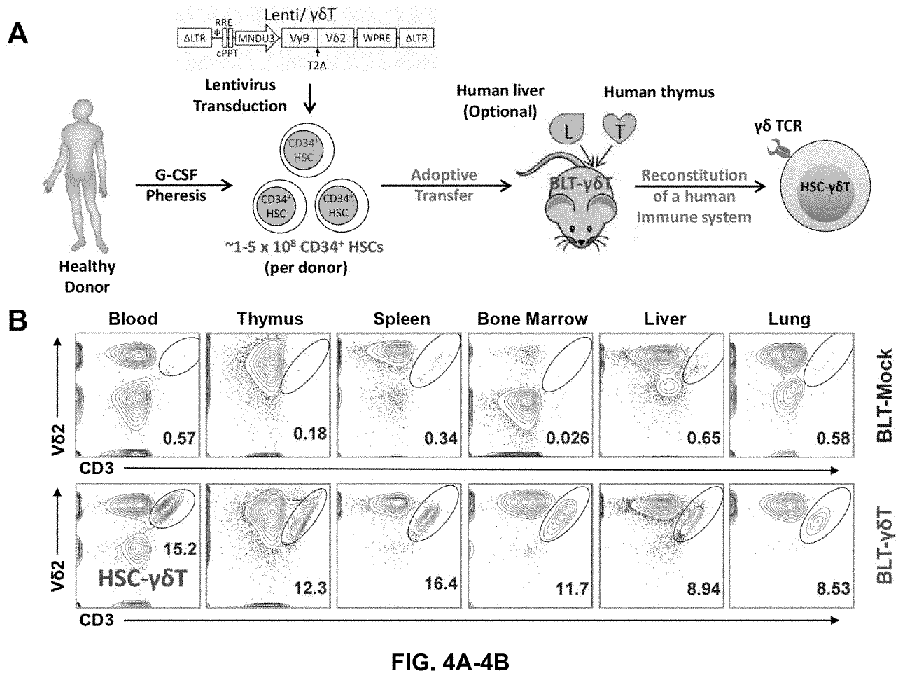

FIG. 4 shows the long-term in vivo provision of transgenic γδT cells through adoptive transfer of γδTCR gene-engineered HSCs. Increasing the number of functional γδT cells in cancer patients may enhance anti-tumor immunity; this can be potentially achieved by adoptively transferring of γδTCR gene engineered autologous HSCs into cancer patients. As shown in FIG. 4, to prove the possibility to generate HSC-engineered γδT cells in vivo, we isolated human CD34⁺ HSCs from G-CSF mobilized healthy donor PBMCs (denoted as PBSCs); transduced with Lenti/γδT vector then adoptively transferred this gene engineered HSCs into a BLT (bone marrow-liver-thymus) humanized mouse model. High numbers (e.g., over 15% of total blood cells) of human HSC-γδT cell were generated in mice and were detected in multiple tissues and organs over a period of 8 weeks. The high levels of transgenic HSC-γδT cells were maintained long-term for over 6 months as long as the experiment ran.

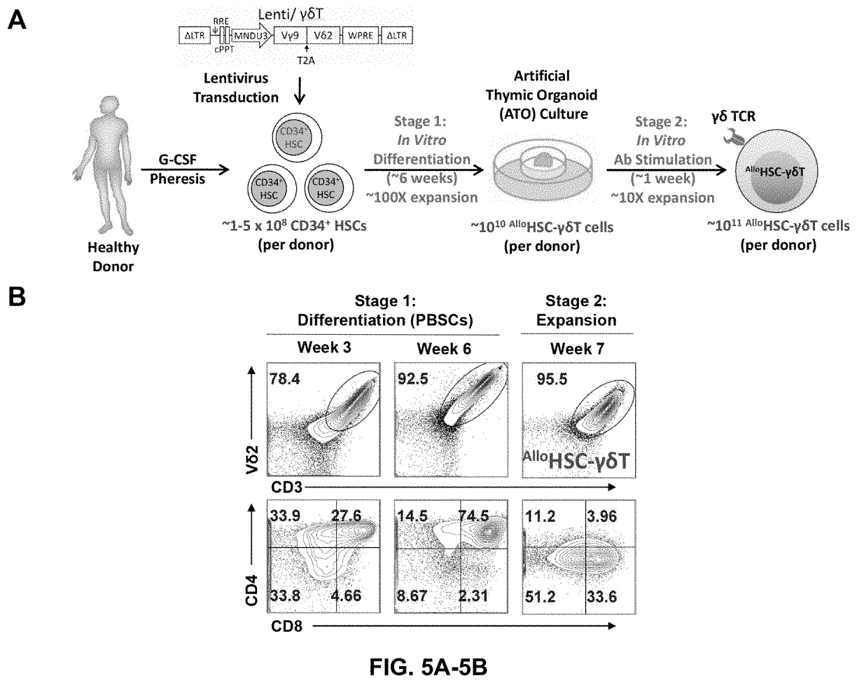

FIG. 5 shows the in vitro Generation of Allogeneic Hematopoietic Stem Cell-Engineered Human γδ T (^{Allo}HSC-γδT) cells (in an Artificial Thymic Organoid (ATO) Culture) for off-the-shelf cell therapy applications. While autologous cell therapy has shown great promise in treating both blood cancers and solid tumors, it is endowed with several limitations. Autologous cells, in particular T cells collected from a patient is time consuming, logistically challenging, and costly; furthermore, patients who undergo heavily lymphopenic pretreatment might not always be possible to produce enough autologous cell products. Allogenic cell products that can be manufactured at large scale and distributed readily to treat a broad range of cancer patients are in great demand. As shown in FIG. 5, embodiments of the invention build on the HSC engineering approach and developed two in vitro culture method (feeder-dependent and feeder-independent cultures) to produce large number of off-the-shelf human γδT cells for allogeneic cell therapy applications.

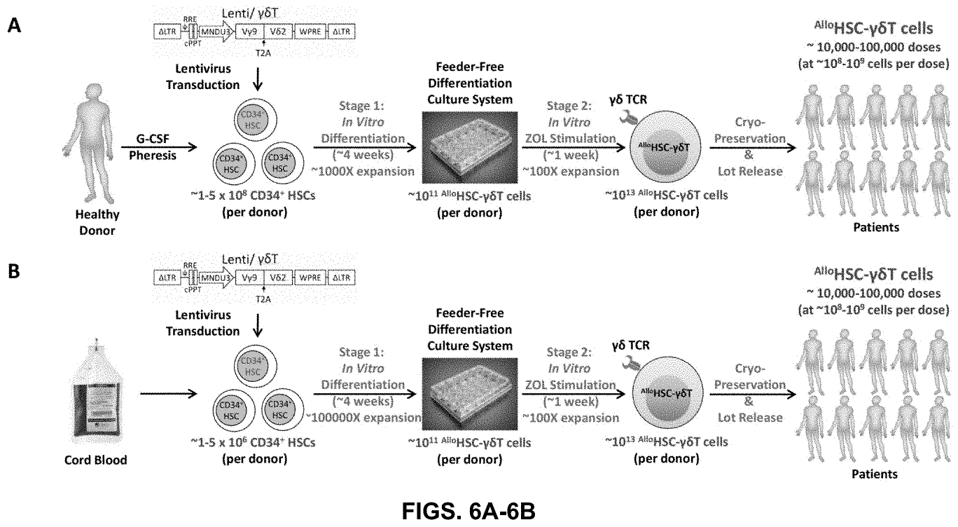

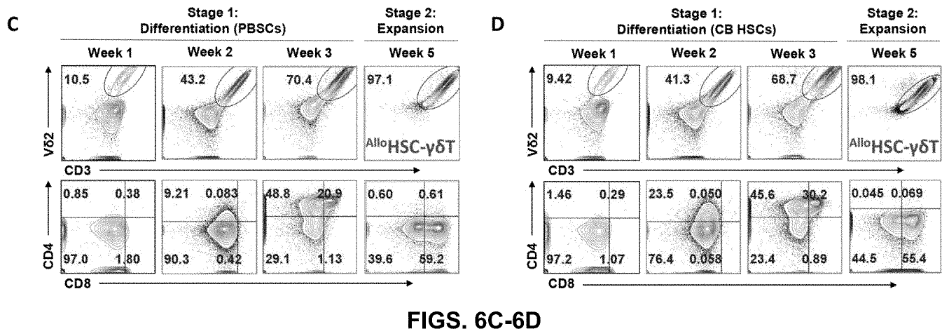

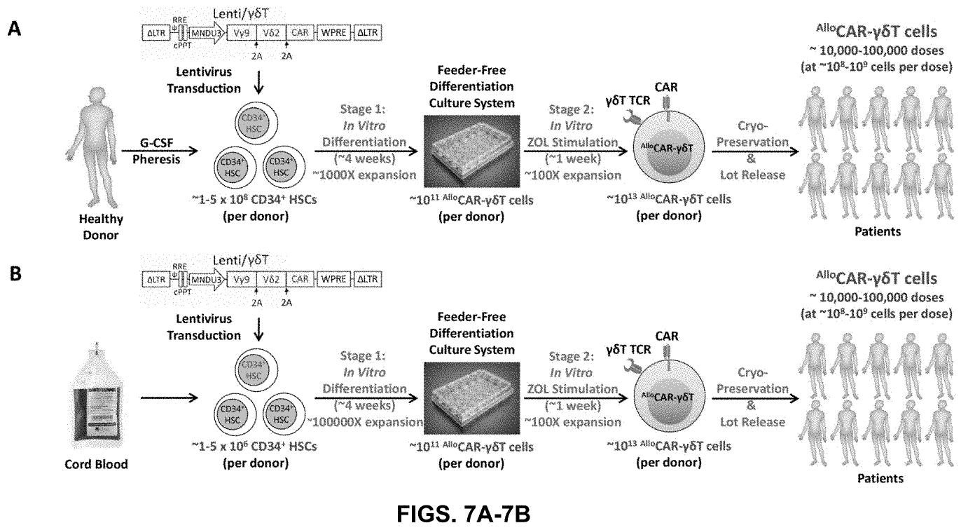

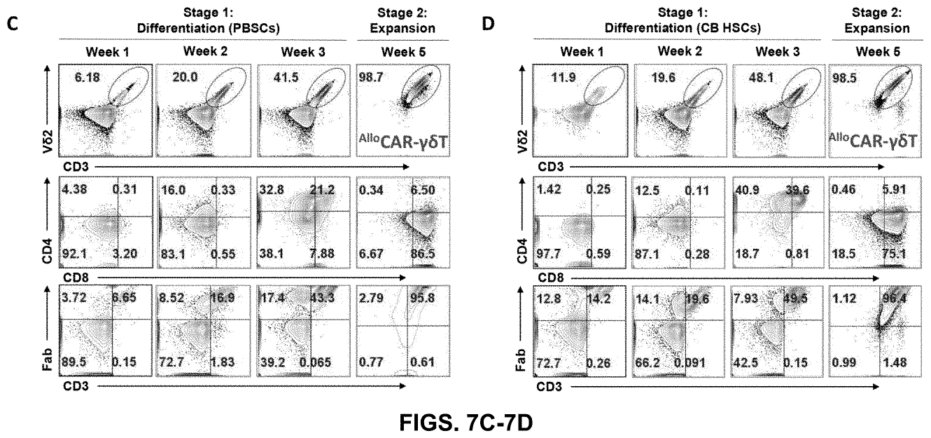

FIG. 6 shows the generation of ^{Allo}HSC-γδT Cells in A Feeder-Free Ex Vivo Differentiation Culture. As shown in FIG. 6. CD34⁺ HSCs isolated from G-CSF-mobilized peripheral blood (denoted as PBSCs) or cord blood (denoted as CB HSCs) were transduced with a Lenti/γδT vector encoding a human γδ TCR gene, then put into the feeder-free ex vivo cell culture to generate ^{Allo}HSC-γδT cells (FIGS. 6A and 6B). Both PBSCs and CB HSCs can effectively differentiate into and expand as transgenic ^{Allo}HSC-γδT cells (FIGS. 6C and 6D). Similarly, ^{Allo}CAR-γδT cells can be generated by transducing the HSCs with a lentiviral vector encoding a human γδ TCR gene together with a CAR gene (FIG. 7). It is estimated that ˜10¹³ scale of ^{Allo}HSC-γδT cells can be produced from either PBSCs of a healthy donor or HSCs of a CB sample, which can be formulated into 10,000-100,000 doses (at ˜10⁸-10⁹ cells per dose) (FIGS. 7A and 7B). Despite the differences in expansion fold, ^{Allo}HSC-γδT cells and their derivatives generated from PBSCs, and CB HSCs displayed similar phenotype and functionality. Unless otherwise indicated, CB HSC-derived ^{Allo}HSC-γδT cells and their derivatives were utilized for the proof-of-principle studies described below. FIG. 7 then shows the generation of ^{Allo}CAR-γδT Cells in A Feeder-Free Ex Vivo Differentiation Culture,

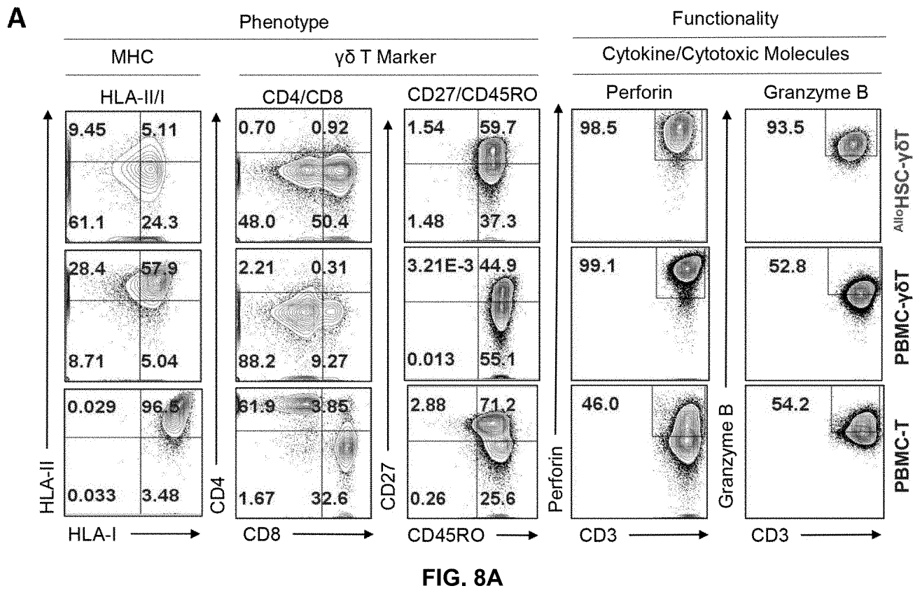

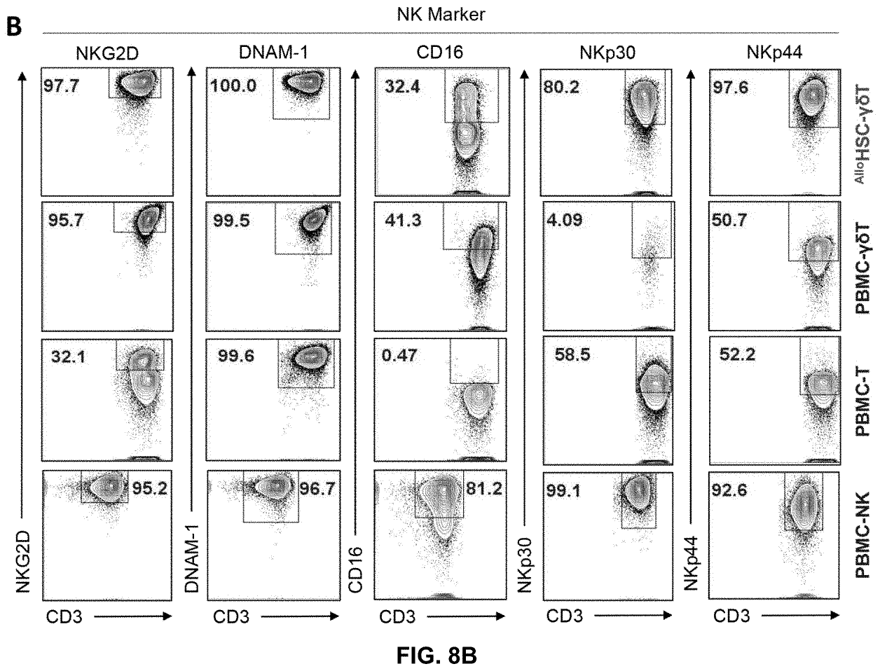

FIG. 8 shows data from a pharmacology Study—^{Allo}HSC-γδT Cells. The phenotype and functionality of ^{Allo}HSC-γδT cells were studied using flow cytometry (FIG. 8). Three controls were included: 1) endogenous human γδ T cells that were isolated from healthy donor peripheral blood (denoted as PBMC-γδ T cells) and expanded in vitro with ZOL stimulation, identified as CD3⁺TCRVδ2⁺; 2) endogenous human conventional αβ T cells that were isolated from healthy donor peripheral blood (denoted as PBMC-T cells) and expanded in vitro with anti-CD3/CD28 stimulation, identified as CD3⁺ TCRαβ⁺; and 3) endogenous human NK cells that were isolated from healthy donor peripheral blood (denoted as PBMC-NK cells) and expanded in vitro with K562 based artificial antigen presenting cell (aAPC) stimulation, identified as CD3⁻-CD56⁺. ^{Allo}HSC-γδT cells produced exceedingly high levels of multiple cytotoxic molecules (e.g., perforin and Granzyme B), and expressed memory T cell marker CD27 and CD45RO, resembling that of endogenous γδ T cells (FIG. 8A). In addition, ^{Allo}HSC-γδT cells expressed high level of NK activation receptors (e.g., NKG2D) and (e.g., DNAM-1) at levels similar to that of endogenous γδ T cells (FIG. 8B). Interestingly, ^{Allo}HSC-γδT cells expressed higher levels of NKp30 and NKp44 (FIG. 8B) than that of endogenous γδ T cells, which suggests that ^{Allo}HSC-γδT cells may have enhanced NK-path tumor killing capacity stronger than that of endogenous γδ T and even endogenous NK cells.

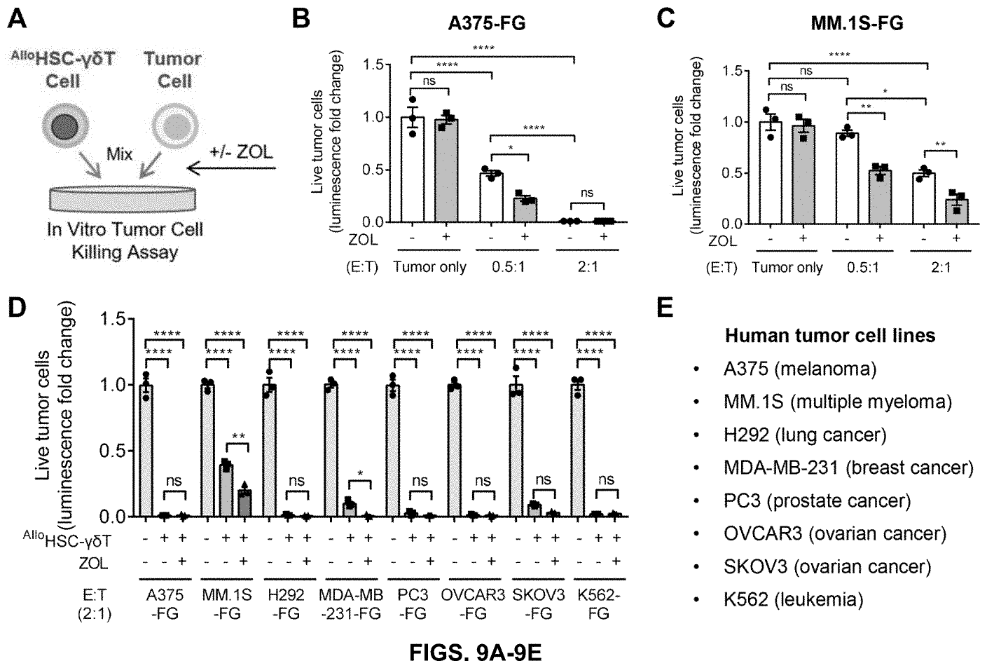

FIG. 9 shows data from an in vitro Efficacy and MOA Study—AlloHSC-γδT Cells. One of the most attractive features of γδ T cells is that they can attack tumors through multiple mechanisms including γδ TCR-mediated and NK receptor-mediated pathways. We therefore established an in vitro tumor cell killing assay to study such tumor killing capacities (FIG. 9A). Human tumor cell lines were engineered to overexpress the firefly luciferase (Fluc) and enhanced green fluorescence protein (EGFP) dual reporters to enable the sensitive measurement of tumor cell killing using luminescence reading or flow cytometry assay. Multiple engineered human tumor cell lines were used in this study as target cells (FIG. 9E), including a melanoma cell line (A375), a multiple myeloma cell line (MM.1S), a lung cancer cell line (H292-FG), a breast cancer cell line (MDA-MB-231), a prostate cancer (PC3-FG), ovarian cancer cell lines (OVCAR3 and OVCAR8), a leukemia cell line (K562). As expected, the ^{Allo}HSC-γδT cells effectively killed the tumor cells through NK pathway on their own and the tumor killing efficacies can be further enhanced by the addition of ZOL, indicating the presence of a γδ TCR-mediated killing mechanism (FIGS. 9B, 9C and 9D).

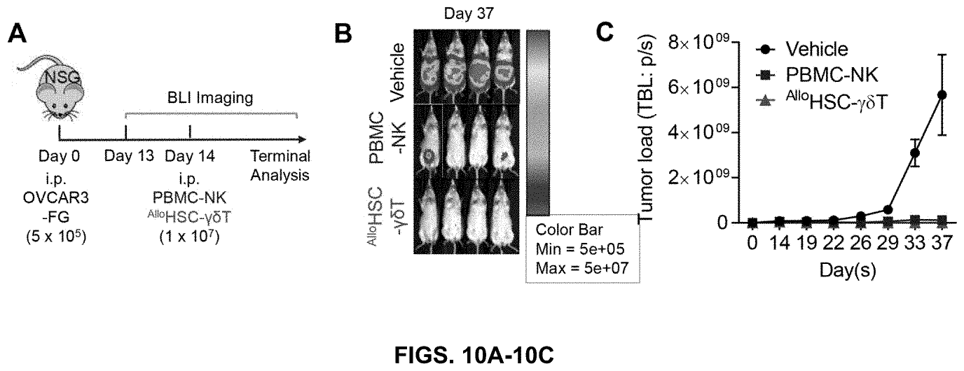

FIG. 10 shows data from an in In Vivo Antitumor Efficacy and MOA Study—^{Allo}HSC-γδT Cells. As shown in FIG. 10, we evaluated the in vivo antitumor efficacy of ^{Allo}HSC-γδT cells using a human ovarian cancer xenograft NSG mouse model. OVCAR3-FG tumor cells were intraperitoneally (i.p.) inoculated into NSG mice to form tumors, followed by an i.p. injection of PBMC-NK or ^{Allo}HSC-γδT cells (FIG. 10A). ^{Allo}HSC-γδT cells effectively suppressed tumor growth at an efficacy similar to or higher than that of PBMC-NK cells, as evidenced by time-course live animal bioluminescence imaging (BLI) monitoring (FIGS. 10B and 10C).

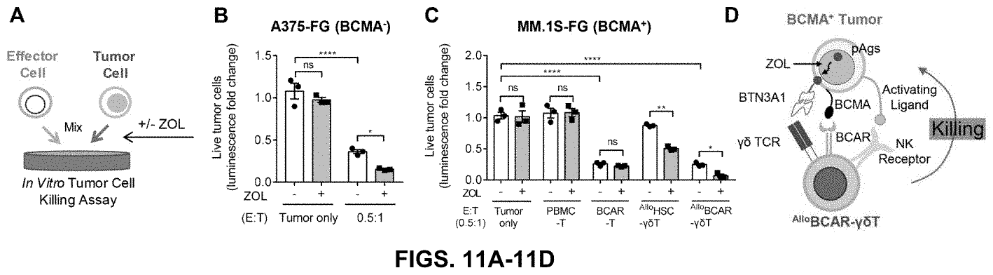

FIG. 11 shows data from an in in vitro Efficacy and MOA Study—^{Allo}BCAR-γδT Cells. As shown in FIG. 11, the tumor attacking potency of allogenic HSC-engineered B cell maturation antigen (BCMA)-targeting CAR armed γδT (^{Allo}BCAR-γδT) cells were studied using the established in vitro tumor killing assay as previously described (FIG. 11A). Two human tumor cell lines were included in this study: 1) a human MM cell line, MM.1 S, which is BCMA⁺ and serves as a target of CAR-mediated killing; and 2) a human melanoma cell line, A375, which is BCMA⁻ and serves as a negative control target of CAR-mediated killing. Both human tumor cell lines were engineered to overexpress the firefly luciferase (Fluc) and enhanced green fluorescence protein (EGFP) dual reporters and the resulting MM.1S-FG and A375-FG cell lines were then utilized in the study. Similar to ^{Allo}HSC-γδT cells, ^{Allo}BCAR-γδT cells killed BCMA⁻ A375-FG cells at certain efficacy, presumably through a CAR-independent NK killing path; tumor killing efficacy was further enhanced in the presence of ZOL, likely through the addition of a gdTCR killing path (FIG. 11B). More importantly, when tested using the BCMA⁺ tumor line MM, ^{Allo}BCAR-γδT cells effectively killed tumor cells, at an efficacy better than that of HSC-γδT and comparable to that of the conventional BCAR-T cells (FIG. 11C). Taken together, these results provide evidence that ^{Allo}BCAR-γδT cells can target human tumor cells using three mechanisms: 1) CAR-dependent path, 2) γδ TCR-dependent path, and 3) NK path (FIG. 11D). This unique triple-targeting capacity of ^{Allo}BCAR-γδT cells is attractive, because it can potentially circumvent antigen escape, a phenomenon that has been reported in autologous CAR-T therapy clinical trials wherein tumor cells down-regulated their expression of CAR-targeting antigen to escape attack from CAR-T cells.

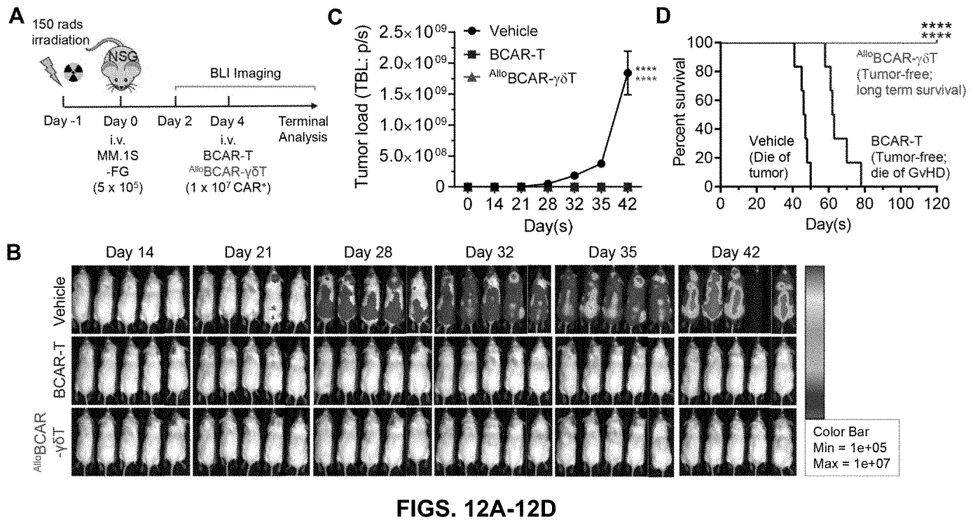

FIG. 12 shows data from an In Vivo Antitumor Efficacy Study—^{Allo}BCAR-γδT Cells. As shown in FIG. 12, the in vivo antitumor efficacy of ^{Allo}BCAR-γδT cells was studied using an established MM.1S-FG xenograft NSG mouse model; conventional BCAR-T cells were included as a control. Under a low-tumor-load condition (FIG. 12A), ^{AO}BCAR-γδT cells eliminated MM tumor cells as effectively as BCAR-T cells (FIGS. 12B and 12D); however, experimental mice treated with BCAR-T cells eventually died of graft-versus-host disease (GvHD) despite being tumor-free, while experimental mice treated with ^{Allo}BCAR-γδT cells lived long-term with tumor-free and GvHD-free (FIG. 12C).

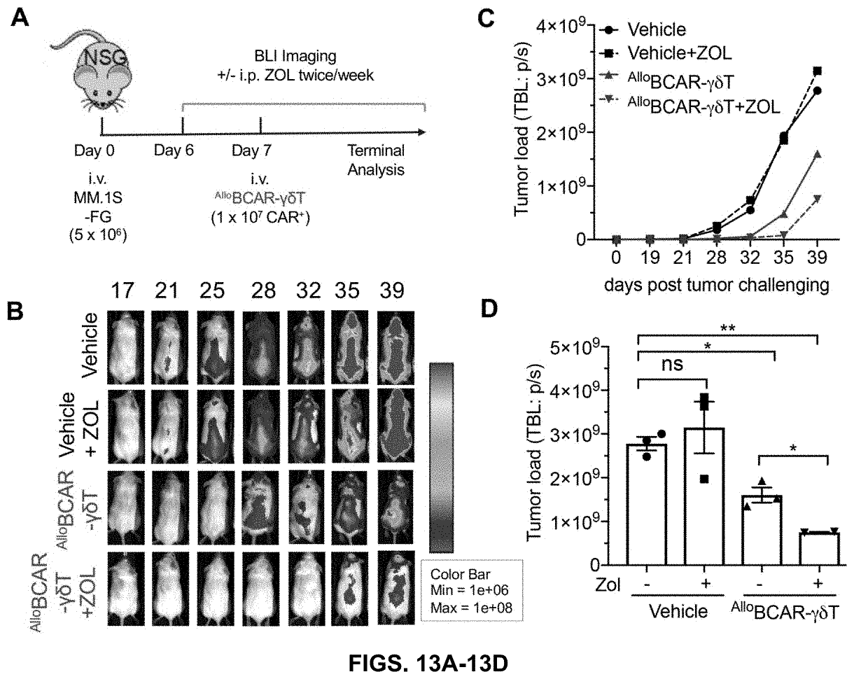

FIG. 13 shows data from an In Vivo Antitumor Efficacy Study—^{Allo}BCAR-γδT Cells combined with ZOL treatment. As shown in FIG. 13, the in vivo antitumor efficacy of ^{Allo}BCAR-γδT cells in combination of ZOL treatment was also studied using an established MN.1S-FG xenograft NSG mouse model under a high-tumor-load condition. ZOL treatment was included to test a possible enhancement of antitumor efficacy of ^{Allo}BCAR-γδT cells through γδ TCR stimulation. ^{Allo}BCAR-γδT cells significantly suppressed tumor growth (FIG. 13A); ZOL treatment further enhanced the efficacy (FIGS. 13B-13D). This result suggests that combining with ZOL treatment may further enhance the antitumor efficacy of ^{Allo}BCAR-γδT cells. Because ZOL is a small molecule drug clinically available, the potential of a ^{Allo}BCAR-γδT cell and ZOL combination therapy is feasible and attractive.

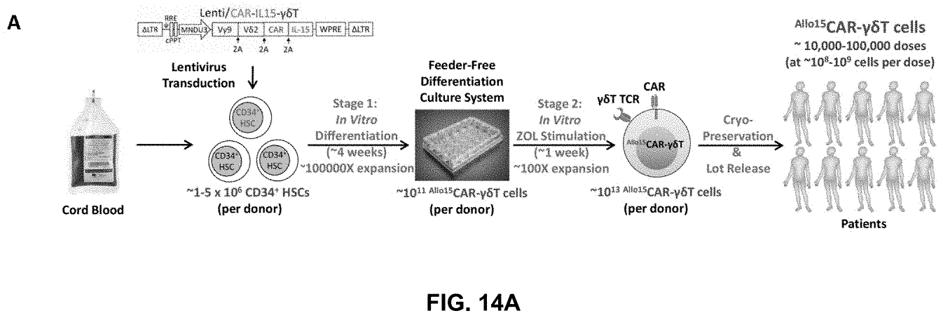

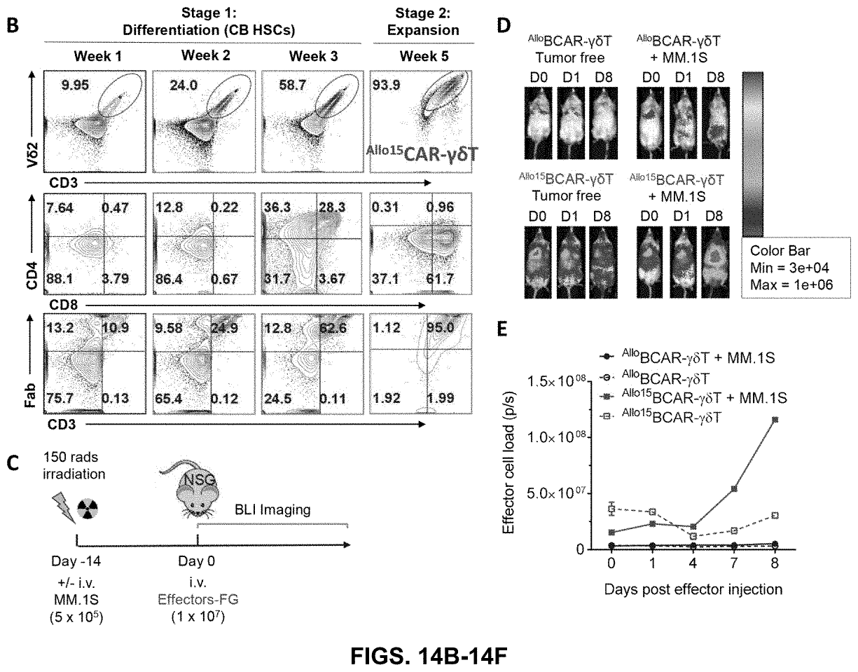

FIG. 14 shows data from studies on the generation and characterization of IL-15-enhanced ^{Allo}BCAR-γδT cells (denoted as ^{Allo15}BCAR-γδT cells). IL-15 is a critical cytokine supporting the in vivo persistence and functionality of many immune cells including many subtypes of T cells and NK cells; we therefore studied the possible benefits of including IL-15 in the ^{Allo}BCAR-T cell product. A Lenti/BCAR-IL15-γδT lentivector was constructed to co-deliver the BCAR, IL-15, and γδ TCR genes (FIG. 14A). CB-derived CD34⁺ HSCs were transduced with the Lenti/BCAR-IL15-γδT vector, then put into the established feeder-free Ex Vivo HSC-γδT Differentiation Culture (FIG. 14A). ^{Allo15}BCAR-γδT cells were produced successfully, following a differentiation path and at a yield similar to that of the basic ^{Allo}BCAR-γδT cells (FIGS. 14A&14B). Importantly, compared to the basic ^{Allo}BCAR-γδT cells, the IL-15-enhanced ^{Allo15}BCAR-γδT cells showed significantly improved in vivo persistence, and when encountering pre-established MM tumors, showed significantly improved antitumor responses (e.g., in vivo clonal expansion; FIGS. 14C-14E).

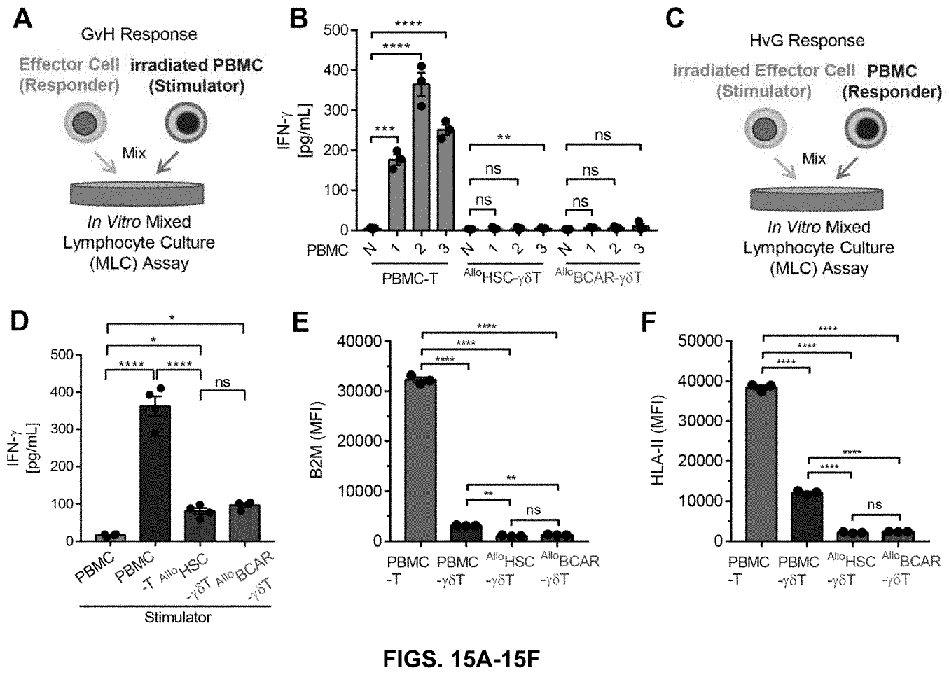

FIG. 15 shows data from an Immunogenicity Study—^{Allo}HSC-γδT and ^{Allo}BCAR-γδT Cells. As shown in FIG. 15, for allogeneic cell therapies, there are two immunogenicity concerns: a) Graft-versus-host (GvH) responses, and b) Host-versus-graft (HvG) responses. GvHD is a major safety concern. However, since γδ T cells do not react to mismatched HLA molecules and protein autoantigens, they are not expected to induce GvHD. This notion is evidenced by the lack of GvHD in human clinical experiences in allogeneic HSC transfer and autologous γδ T cell transfer and is supported by our in vitro mixed lymphocyte culture (MLC) assay (FIG. 15A). Note that neither PBMC-γδ T cells nor ^{Allo}HSC-γδT cells respond to allogenic PBMCs, in sharp contrast to that of the conventional PBMC-T cells (FIG. 15B). On the other hand, HvG risk is largely an efficacy concern, mediated through elimination of allogeneic therapeutic cells by host immune cells, mainly by conventional CD8 and CD4 αβ T cells which recognize mismatched HLA-I and HLA-II molecules. Indeed, in an In Vitro Mixed Lymphocyte Culture (MLC) assay (FIG. 15C), both conventional PBMC-T and PBMC-γδT cells triggered significantly responses from the PBMC-T cells of multiple mismatched donors (FIG. 15D). Interestingly, ^{Allo}HSC-γδT cells showed reduced immunogenicity, likely attributes to their low expression levels of HLA-I/II (FIGS. 15E and 15F). Taken together, these results strongly support ^{Allo}HSC-γδT cells as an ideal candidate for off-the-shelf cellular therapy that are GvHD-free and HvG-resistant.

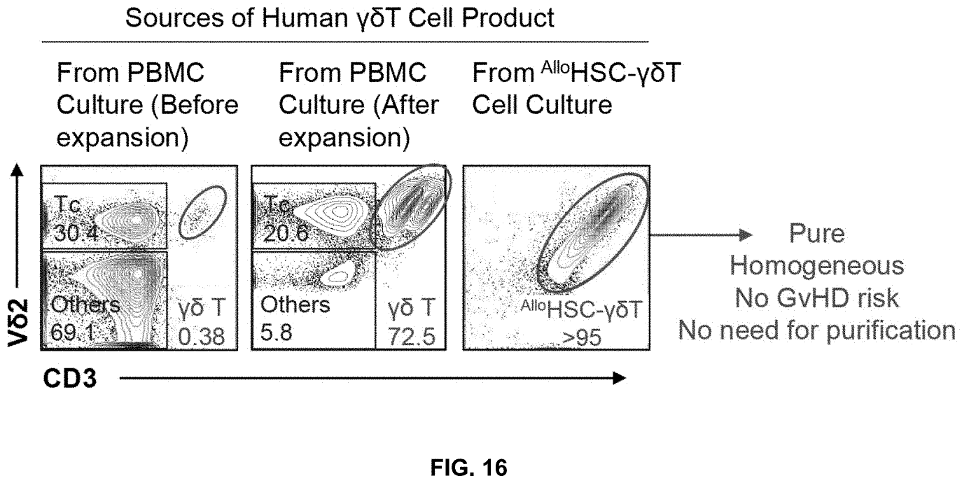

FIG. 16 provides data from a comparison Study—Unique Properties of ^{Allo}HSC-γδT Cell Products. Existing methods generating human γδ T cell products mainly reply on expanding γδ T cells from human PBMCs. This culture method starts and ends up with a mixed cell population containing human γδ T cells as well as other cells, in particular heterogeneous conventional up T (Tc) cells that may cause GvHD when transferred into allogeneic recipients (FIG. 16). As a result, this method requires a purification step to make “off-the-shelf” γδT cell products, in order to avoid GvHD. Herein, the ^{Allo}HSC-γδT cell culture is unique in two aspects: 1) It does not support the generation of randomly rearranged αβTCR recombinations to produce randomly rearranged endogenous αβTCRs, thereby no GvHD risk; 2) It supports the synchronized differentiation of transgenic ^{Allo}HSC-γδT cells, thereby eliminating the presence of un-differentiated progenitor cells and other lineages of immune cells. As a result, the ^{Allo}HSC-γδT cell product is pure, homogenous, of no GvHD risk, and therefore no need in this methodology for a cell purification/sorting step.

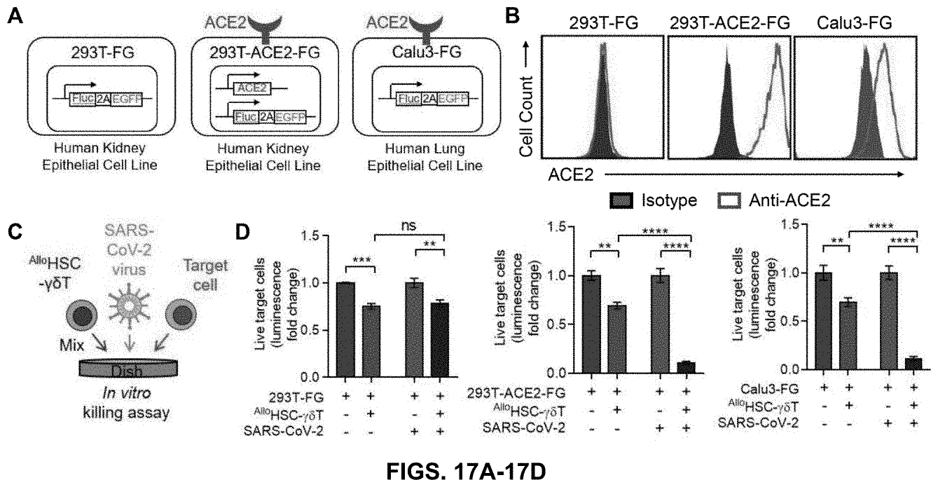

We established an in vitro SARS-CoV-2 infection model (FIGS. 17A-D), to explore the therapeutic potential of ^{Allo}HSC-γδT cells against COVID-19. SARS-CoV-2 mainly enters a host human cell by binding to cell surface ACE2 (Angiotensin-converting enzyme 2) using the virus spike (S) protein; we therefore used two ACE-2-positive human cells as target cells: one is a 293T human epithelial cell line engineered to overexpress ACE2, the other is a Calu-3 human lung epithelial cell line naturally expressed ACE2 (FIG. 17A, 13). These cell lines were further engineered to overexpress firefly luciferase and enhanced green fluorescent protein dual-reporters (FG) to enable the sensitive measurement of cell viability using luminescence reading (FIG. 17A). The ^{Allo}HSC-γδT cells effectively killed both 293T-ACE2-FG and Calu-3-FG target cells with SARS-CoV-2 infection; target cell killing was not observed without virus infection (FIG. 17D). Notably, SARS-CoV-2 infection alone did not affect the viability of the ACE2-positive target cells (FIG. 17D).

It is specifically noted that any embodiment discussed in the context of a particular cell or cell population embodiment may be employed with respect to any other cell or cell population embodiment. Moreover, any embodiment employed in the context of a specific method may be implemented in the context of any other methods described herein. Furthermore, aspects of different methods described herein may be combined so as to achieve other methods, as well as to create or describe the use of any cells or cell populations. It is specifically contemplated that aspects of one or more embodiments may be combined with aspects of one or more other embodiments described herein. Furthermore, any method described herein may be phrased to set forth one or more uses of cells or cell populations described herein. For instance, use of engineered γδ T cells or a γδ T cell population can be set forth from any method described herein.

In a particular embodiment, there is an engineered γδ T cell that expresses at least one γδ T-cell receptor (γδ TCR) and an exogenous suicide gene product, wherein the at least one γδ TCR is expressed from an exogenous nucleic acid and/or from an endogenous γδ TCR gene that is under the transcriptional control of a recombinantly modified promoter region. Methods in the art for suicide gene usage may be employed, such as in U.S. Pat. No. 8,628,767, U.S. Patent Application Publication 20140369979, U.S. 20140242033, and U.S. 20040014191, all of which are incorporated by reference in their entirety. In further embodiments, a TK gene is a viral TK gene, i.e., a TK gene from a virus. In particular embodiments, the TK gene is a herpes simplex virus TK gene. In some embodiments, the suicide gene product is activated by a substrate. Thymidine kinase is a suicide gene product that is activated by ganciclovir, penciclovir, or a derivative thereof. In certain embodiments, the substrate activating the suicide gene product is labeled in order to be detected. In some instances, the substrate that may be labeled for imaging. In some embodiments, the suicide gene product may be encoded by the same or a different nucleic acid molecule encoding one or both of TCR-gamma or TCR-delta. In certain embodiments, the suicide gene is sr39TK or inducible caspase 9. In alternative embodiments, the cell does not express an exogenous suicide gene.

In additional embodiments, an engineered γδ T cell is lacking or has reduced surface expression of at least one HLA-I or HLA-II molecule. In some embodiments, the lack of surface expression of HLA-I and/or HLA-II molecules is achieved by disrupting the genes encoding individual HLA-I/II molecules, or by disrupting the gene encoding B2M (beta 2 microglobulin) that is a common component of all HLA-I complex molecules, or by disrupting the genes encoding CIITA (the class II major histocompatibility complex transactivator) that is a critical transcription factor controlling the expression of all HLA-II genes. In specific embodiments, the cell lacks the surface expression of one or more HLA-I and/or HLA-II molecules, or expresses reduced levels of such molecules by (or by at least) 50, 60, 70, 80, 90, 100% (or any range derivable therein). In some embodiments, the HLA-I or HLA-II are not expressed in the γδ T cell because the cell was manipulated by gene editing. In some embodiments, the gene editing involved is CRISPR-Cas9. Instead of Cas9, CasX or CasY may be involved. Zinc finger nuclease (ZFN) and TALEN are other gene editing technologies, as well as Cpf1, all of which may be employed. In other embodiments, the γδ T cell comprises one or more different siRNA or miRNA molecules targeted to reduce expression of HLA-I/II molecules, B2M, and/or CIITA.

In some embodiments, a γδ T cell of the invention comprises a recombinant vector or a nucleic acid sequence from a recombinant vector that was introduced into the cells. In certain embodiments the recombinant vector is or was a viral vector. In further embodiments, the viral vector is or was a lentivirus, a retrovirus, an adeno-associated virus (AAV), a herpesvirus, or adenovirus. It is understood that the nucleic acid of certain viral vectors integrate into the host genome sequence.

In some embodiments, a γδ T cell of the invention is disposed in selected media conditions during growth and differentiation (e.g., not disposed in media comprising animal serum). In further embodiments, a γδ T cell is or was frozen. In some embodiments, the γδ T cell has previously been frozen and the previously frozen cell is stable at room temperature for at least one hour. In some embodiments, the γδ T cell has previously been frozen and the previously frozen cell is stable at room temperature for at least 1, 2, 3, 4, 5, 6, 7, 8, 10, 15, 20, 24, 30, or 48 hours (or any derivable range therein). In certain embodiments, a γδ T cell or a population of γδ T cells in a solution comprises dextrose, one or more electrolytes, albumin, dextran, and/or DMSO. In a further embodiment, the cell is in a solution that is sterile, nonpyogenic, and isotonic.

In embodiments involving multiple cells, a γδ T cell population may comprise, comprise at least, or comprise at most about 10², 10³, 10⁴′, 10⁵, 10⁶, 10⁷, 10⁸, 10⁹, 10¹⁰, 10¹¹, 10¹², 10¹³, 10¹⁴, 10¹⁵ cells or more (or any range derivable therein), which are engineered γδ T cells in some embodiments. In some cases, a cell population comprises at least about 10⁶-10¹² engineered γδ T cells. It is contemplated that in some embodiments, that a population of cells with these numbers is produced from a single batch of cells and are not the result of pooling batches of cells separately produced.

In specific embodiments, there is an T cell population comprising: clonal γδ T cells comprising one or more exogenous nucleic acids encoding an γδ T-cell receptor and a thymidine kinase suicide gene product, wherein the clonal γδ T cells have been engineered not to express functional beta-2-microglobulin (B2M), and/or class II, major histocompatibility complex, or transactivator (CIITA) and wherein the cell population is at least about 10⁶-10¹² total cells and comprises at least about 10²-10⁶ engineered γδ T cells. In certain instances, the cells are frozen in a solution.

A number of embodiments concern methods of preparing an γδ T cell or a population of cells, particularly a population in which some are all the cells are clonal. In certain embodiments, a cell population comprises cells in which at least or at most 50%, 55%, 60%, 65%, 70%, 75%, 80%, 85%, 90%, 95%, 96%, 97%, 98%, 99%, 100% (or any range derivable therein) of the cells are clonal, i.e., the percentage of cells that have been derived from the same ancestor cell as another cell in the population. In other embodiments, a cell population comprises a cell population that is comprised of cells arising from, from at least, or from at most 1, 2, 3, 4, 5, 6, 7, 8, 9, 10, 11, 12, 13, 14, 15, 16, 17, 18, 19, 20, 21, 22, 23, 24, 25, 26, 7, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 (or any range derivable therein) different parental cells.

Methods for preparing, making, manufacturing, and using engineered γδ T cells and γδ T cell populations are provided. Methods include 1, 2, 3, 4, 5, 6, 7, 8, 9, 10, 11, 12, 13, 14, 15 or more of the following steps in embodiments: obtaining pluripotent cells; obtaining hematopoietic progenitor cells; obtaining progenitor cells capable of becoming one or more hematopoietic cells; obtaining progenitor cells capable of becoming γδ T cells; selecting cells from a population of mixed cells using one or more cell surface markers; selecting CD34⁺ cells from a population of cells; isolating CD34⁺ cells from a population of cells; separating CD34⁺ and CD34⁻ cells from each other; selecting cells based on a cell surface marker other than or in addition to CD34; introducing into cells one or more nucleic acids encoding an γδ T-cell receptor (TCR); infecting cells with a viral vector encoding an γδ T-cell receptor (TCR); transfecting cells with one or more nucleic acids encoding an γδ T-cell receptor (TCR); transfecting cells with an expression construct encoding an γδ T-cell receptor (TCR); integrating an exogenous nucleic acid encoding an γδ T-cell receptor (TCR) into the genome of a cell; introducing into cells one or more nucleic acids encoding a suicide gene product; infecting cells with a viral vector encoding a suicide gene product; transfecting cells with one or more nucleic acids encoding a suicide gene product; transfecting cells with an expression construct encoding a suicide gene product; integrating an exogenous nucleic acid encoding a suicide gene product into the genome of a cell; introducing into cells one or more nucleic acids encoding one or more polypeptides and/or nucleic acid molecules for gene editing; infecting cells with a viral vector encoding one or more polypeptides and/or nucleic acid molecules for gene editing; transfecting cells with one or more nucleic acids encoding one or more polypeptides and/or nucleic acid molecules for gene editing; transfecting cells with an expression construct encoding one or more polypeptides and/or nucleic acid molecules for gene editing; integrating an exogenous nucleic acid encoding one or more polypeptides and/or nucleic acid molecules for gene editing; editing the genome of a cell; editing the promoter region of a cell; editing the promoter and/or enhancer region for an γδ TCR gene; eliminating the expression one or more genes; eliminating expression of one or more HLA-I/II genes in the isolated human CD34⁺ cells; transfecting into a cell one or more nucleic acids for gene editing; culturing isolated or selected cells; expanding isolated or selected cells; culturing cells selected for one or more cell surface markers; culturing isolated CD34⁺ cells expressing γδ TCR; expanding isolated CD34⁺ cells; culturing cells under conditions to produce or expand γδ T cells; culturing cells in an artificial thymic organoid (ATO) system to produce γδ T cells; culturing cells in serum-free medium; culturing cells in an ATO system, wherein the ATO system comprises a 3D cell aggregate comprising a selected population of stromal cells that express a Notch ligand and a serum-free medium. It is specifically contemplated that one or more steps may be excluded in an embodiment.

In some embodiments, there are methods of preparing a population of clonal γδ T cells comprising: a) selecting CD34⁺ cells from human peripheral blood cells (PBMCs); b) introducing one or more nucleic acids encoding a human γδ T-cell receptor (TCR); c) eliminating surface expression of one or more HLA-I/II genes in the isolated human CD34⁺ cells; and, d) culturing isolated CD34⁺ cells expressing γδ TCR (e.g. in an artificial thymic organoid system) to produce γδ T cells. Typically, the ATO system comprises a 3D cell aggregate comprising a selected population of stromal cells that express a Notch ligand and a serum-free medium.

Pluripotent cells that may be used to create engineered γδ T cells include CD34⁺ hematopoietic progenitor stem cells. Cells may be from peripheral blood mononuclear cells (PBMCs), bone marrow cells, fetal liver cells, embryonic stem cells, cord blood cells, induced pluripotent stem cells (iPS cells), or a combination thereof. In some embodiments, methods comprise isolating CD34⁻ cells or separating CD34⁻ and CD34⁺ cells. While embodiments involve manipulating the CD34⁺ cells further, CD34⁻ cells may be used in the creation of γδ T cells. Therefore, in some embodiments, the CD34⁻ cells are subsequently used, and may be saved for this purpose.

Certain methods involve culturing selected CD34⁺ cells in media prior to introducing one or more nucleic acids into the cells. Culturing the cells can include incubating the selected CD34⁺ cells with media comprising one or more growth factors. In some embodiments, one or more growth factors comprise c-kit ligand, flt-3 ligand, and/or human thrombopoietin (TPO). In further embodiments, the media includes c-kit ligand, flt-3 ligand, and TPO. In some embodiments, the concentration of the one or more growth factors is between about 5 ng/ml to about 500 ng/ml with respect to either each growth factor or the total of any and all of these particular growth factors. The concentration of a single growth factor or the combination of growth factors in media can be about, at least about, or at most about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500 (or any range derivable) ng/ml or g/ml or more.

In typical embodiments, a nucleic acid may comprise a nucleotide sequence encoding an γ-TCR and/or a S-TCR, as discussed herein. In certain embodiments, one nucleic acid encodes both the gamma and delta chains of the TCR. In some embodiments, a further nucleic acid may comprise a nucleic acid sequence encoding an α-TCR and/or a β-TCR polypeptide, and/or one or more iNKT TCR polypeptides. In additional embodiments, a nucleic acid further comprises a nucleic acid sequence encoding a suicide gene product. In some embodiments, a nucleic acid molecule that is introduced into a selected CD34⁺ cell encodes the TCR, and the suicide gene product. In other embodiments, a method also involves introducing into the selected CD34⁺ cells a nucleic acid encoding a suicide gene product, in which case a different nucleic acid molecule encodes the suicide gene product than a nucleic acid encoding at least one of the TCR genes.

As discussed above, in some embodiments the γδ T cells do not express the HLA-I and/or HLA-II molecules on the cell surface, which may be achieved by disrupting the expression of genes encoding beta-2-microglobulin (B2M), transactivator (CIITA), or HLA-I and HLA-II molecules. In certain embodiments, methods involve eliminating surface expression of one or more HLA-I/II molecules in the isolated human CD34⁺ cells. In particular embodiments, eliminating expression may be accomplished through gene editing of the cell's genomic DNA. Some methods include introducing CRISPR and one or more guide RNAs (gRNAs) corresponding to B2M or CIITA into the cells. In particular embodiments, CRISPR or the one or more gRNAs are transfected into the cell by electroporation or lipid-mediated transfection. Consequently, methods may involve introducing CRISPR and one or more gRNAs into a cell by transfecting the cell with nucleic acid(s) encoding CRISPR and the one or more gRNAs. A different gene editing technology may be employed in some embodiments.

Similarly, in some embodiments, one or more nucleic acids encoding the TCR receptor are introduced into the cell. This can be done by transfecting or infecting the cell with a recombinant vector, which may or may not be a viral vector as discussed herein. The exogenous nucleic acid may incorporate into the cell's genome in some embodiments.

In some embodiments, cells are cultured in cell-free medium. In certain embodiments, the serum-free medium further comprises externally added ascorbic acid. In particular embodiments, methods involve adding ascorbic acid medium. In further embodiments, the serum-free medium further comprises 1, 2, 3, 4, 5, 6, 7, 8, 9, 10, 11, 12, 13, 14, 15, or all 16 (or a range derivable therein) of the following externally added components: FLT3 ligand (FLT3L), interleukin 7 (IL-7), stem cell factor (SCF), thrombopoietin (TPO), stem cell factor (SCF), IL-2, IL-4, IL-6, IL-15, IL-21, TNF-alpha, TGF-beta, interferon-gamma, interferon-lambda, TSLP, thymopentin, pleotrophin, or midkine. In additional embodiments, the serum-free medium comprises one or more vitamins. In some cases, the serum-free medium includes 1, 2, 3, 4, 5, 6, 7, 8, 9, 10, 11, or 12 of the following vitamins (or any range derivable therein): comprise biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or a salt thereof. In certain embodiments, medium comprises or comprise at least biotin, DL alpha tocopherol acetate, DL alpha-tocopherol, vitamin A, or combinations or salts thereof. In additional embodiments, serum-free medium comprises one or more proteins. In some embodiments, serum-free medium comprises 1, 2, 3, 4, 5, 6 or more (or any range derivable therein) of the following proteins: albumin or bovine serum albumin (BSA), a fraction of BSA, catalase, insulin, transferrin, superoxide dismutase, or combinations thereof. In other embodiments, serum-free medium comprises 1, 2, 3, 4, 5, 7, 8, 9, 10, or 11 of the following compounds: corticosterone, D-Galactose, ethanolamine, glutathione, L-carnitine, linoleic acid, linolenic acid, progesterone, putrescine, sodium selenite, or triodo-I-thyronine, or combinations thereof. In further embodiments, serum-free medium comprises a B-27® supplement, xeno-free B-27® supplement, GS21TM supplement, or combinations thereof. In additional embodiments, serum-free medium comprises or further comprises amino acids, monosaccharides, and/or inorganic ions. In some aspects, serum-free medium comprises 1, 2, 3, 4, 5, 6, 7, 8, 9, 10, 11, 12, or 13 of the following amino acids: arginine, cysteine, isoleucine, leucine, lysine, methionine, glutamine, phenylalanine, threonine, tryptophan, histidine, tyrosine, or valine, or combinations thereof. In other aspects, serum-free medium comprises 1, 2, 3, 4, 5, or 6 of the following inorganic ions: sodium, potassium, calcium, magnesium, nitrogen, or phosphorus, or combinations or salts thereof. In additional aspects, serum-free medium comprises 1, 2, 3, 4, 5, 6 or 7 of the following elements: molybdenum, vanadium, iron, zinc, selenium, copper, or manganese, or combinations thereof.

In some methods, cells are cultured in an artificial thymic organoid (ATO) system. The ATO system involves a three-dimensional (3D) cell aggregate, which is an aggregate of cells. In certain embodiments, the 3D cell aggregate comprises a selected population of stromal cells that express a Notch ligand. In some embodiments, a 3D cell aggregate is created by mixing CD34⁺ transduced cells with the selected population of stromal cells on a physical matrix or scaffold. In further embodiments, methods comprise centrifuging the CD34⁺ transduced cells and stromal cells to form a cell pellet that is placed on the physical matrix or scaffold. In certain embodiments, stromal cells express a Notch ligand that is an intact, partial, or modified DLL1, DLL4, JAG1, JAG2, or a combination thereof. In further embodiments, the Notch ligand is a human Notch ligand. In other embodiments, the Notch ligand is human DLL1.

The methods of the disclosure may produce a population of cells (e.g. via a differentiation and/or expansion step) comprising at least 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, 1×10²⁰, or 1×10²¹ (or any derivable range therein) cells that may express a marker or have a high or low level of a certain marker. The cell population number may be one that is achieved without cell sorting based on marker expression or without cell sorting based on γδ T cell marker expression or without cell sorting based on T-cell marker expression. In some embodiments, the cell population size may be one that is achieved without cell sorting based on the binding of an antigen to a heterologous targeting element, such as a CAR, TCR, BiTE, or other heterologous tumor-targeting agent. Furthermore, the population of cells achieved may be one that comprises at least 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, 1×10²⁰, or 1×10²¹ (or any derivable range therein) cells that is made within a certain time period such as a time period that is at least, at most, or exactly 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 days or 7, 8, 9, 10, 11, 12, 13, 14, 15, 16, 17, 18, 19, 20, 21, 22, 23, 24, 25, 26, 27, 28, 29 or 30 weeks (or any derivable range therein).

In some embodiments, feeder cells used in methods comprise CD34⁻ cells. These CD34⁻ cells may be from the same population of cells selected for CD34⁺ cells. In additional embodiments, cells may be activated. In certain embodiments, methods comprise activating γδ T cells. In specific embodiments, γδ T cells have been activated and expanded with ZOL. Cells may be incubated or cultured with ZOL so as to activate and expand them. In some embodiments, feeder cells have been pulsed with ZOL.

Cells may be used immediately, or they may be stored for future use. In certain embodiments, cells that are used to create γδ T cells are frozen, while produced γδ T cells may be frozen in some embodiments. In some aspects, cells are in a solution comprising dextrose, one or more electrolytes, albumin, dextran, and DMSO. In other embodiments, cells are in a solution that is sterile, nonpyrogenic, and isotonic. In some embodiments, the engineered γδ T cell is derived from a hematopoietic stem cell. In some embodiments, the engineered γδ T cell is derived from a G-CSF mobilized CD34⁺ cells. In some embodiments, the cell is derived from a cell from a human patient that doesn't have cancer. In some embodiments, the cell doesn't express an endogenous TCR.

The number of cells produced by a production cycle may be about, at least about, or at most about 10², 10³, 10⁴, 10⁵, 10⁶, 10⁷, 10⁸, 10⁹, 10¹⁰, 10¹¹, 10¹², 10¹³, 10¹⁴, 10¹⁵ cells or more (or any range derivable therein), which are engineered γδ T cells in some embodiments. In some cases, a cell population comprises at least about 10⁶-10¹² engineered γδ T cells. It is contemplated that in some embodiments, that a population of cells with these numbers is produced from a single batch of cells and are not the result of pooling batches of cells separately produced—i.e., from a single production cycle. In some embodiments, a cell population is frozen and then thawed. The cell population may be used to create engineered γδ T cells, or they may comprise engineered γδ T cells.

In some embodiments, methods include introducing one or more additional nucleic acids into the cell population, which may or may not have been previously frozen and thawed. This use provides one of the advantages of creating an off-the-shelf γδ T cell. In particular embodiments, the one or more additional nucleic acids encode one or more therapeutic gene products. Examples of therapeutic gene products include at least the following: 1. Antigen recognition molecules, e.g. CAR (chimeric antigen receptor) and/or TCR (T cell receptor); 2. Co-stimulatory molecules, e.g. CD28, 4-1BB, 4-1BBL, CD40, CD40L, ICOS; and/or 3. Cytokines, e.g. IL-1α, IL-1β, IL-2, IL-4, IL-6, IL-7, IL-9, IL-15, IL-12, IL-17, IL-21, IL-23, IFN-γ, TNF-α, TGF-β, G-CSF, GM-CSF; 4. Transcription factors, e.g. T-bet, GATA-3, RORγt, FOXP3, and Bcl-6. Therapeutic antibodies are included, as are chimeric antigen receptors, single chain antibodies, monobodies, humanized, antibodies, bi-specific antibodies, single chain FV antibodies or combinations thereof.

In some embodiments, there are engineered γδ T cells produced by a method comprising: a) selecting CD34⁺ cells from human peripheral blood cells (PBMCs); b) culturing the CD34⁺ cells with medium comprising growth factors such as c-kit ligand, flt-3 ligand, and human thrombopoietin (TPO) or the like; c) transducing the selected CD34⁺ cells with a lentiviral vector comprising a nucleic acid sequence encoding γ-TCR, δ-TCR, thymidine kinase, and a reporter gene product; d) introducing into the selected CD34⁺ cells Cas9 and gRNA for beta 2 microglobulin (B2M) and/or CTIIA to eliminate expression of B2M or CTIIA; e) culturing the transduced cells for 2-10 weeks with an irradiated stromal cell line expressing an exogenous Notch ligand to expand γδ T cells in a 3D aggregate cell culture; f) selecting γδ T cells lacking expression of B2M and/or CTIIA; and, g) culturing the selected γδ T cells with irradiated feeder cells.

In particular embodiments, γδ T cells produced from transduced cells (e.g. HSPCs) are further modified to have one or more characteristics, including to render the cells suitable for allogeneic use or more suitable for allogeneic use than if the cells were not further modified to have one or more characteristics. The present disclosure encompasses ^{U}HSC-γδ T cells that are suitable for allogeneic use, if desired. In some embodiments, the HSC-γδ T cells are non-alloreactive and express an exogenous gamma delta TCR. These cells are useful for “off the shelf” cell therapies and do not require the use of the patient's own γδ T or other cells. Therefore, the current methods provide for a more cost-effective, less labor-intensive cell immunotherapy.

In specific embodiments, HSC-γδ T cells are engineered to be HLA-negative to achieve safe and successful allogeneic engraftment without causing graft-versus-host disease (GvHD) and being rejected by host immune cells (HvG rejection). In specific embodiments, allogeneic HSC-γδ T cells do not express endogenous TCRs and do not cause GvHD, because the expression of the transgenic γδ TCR gene blocks the recombination of endogenous TCRs through allelic exclusion. In particular embodiments, allogeneic ^{U}HSC-γδ T cells do not express HLA-I and/or HLA-II molecules on cell surface and resist host CD8⁺ and CD4⁺ T cell-mediated allograft depletion and sr39TK immunogen-targeting depletion. Thus, in certain embodiments the engineered γδ T cells do not express surface HLA-I or -II molecules, achieved through disruption of genes encoding proteins relevant to HLA-I/II expression, including but not limited to beta-2⁻ microglobulin (B2M), major histocompatibility complex II transactivator (CIITA), or HLA-I/II molecules. In some cases, the HLA-I or HLA-II are not expressed on the surface of γδ T cells because the cells were manipulated by gene editing, which may or may not involve CRISPR-Cas9.

In cases wherein the γδ T cells have been modified to exhibit one or more characteristics of any kind, the γδ T cells may comprise nucleic acid sequences from a recombinant vector that was introduced into the cells. The vector may be a non-viral vector, such as a plasmid, or a viral vector, such as a lentivirus, a retrovirus, an adeno-associated virus (AAV), a herpesvirus, or adenovirus.

The γδ T cells of the invention may or may not have been exposed to one or more certain conditions before, during, or after their production. In specific cases, the cells are not or were not exposed to media that comprises animal serum. The cells may be frozen. The cells may be present in a solution comprising dextrose, one or more electrolytes, albumin, dextran, and/or DMSO. Any solution in which the cells are present may be a solution that is sterile, nonpyogenic, and isotonic. The cells may have been activated and expanded by any suitable manner, such as activated with ZOL, for example.

Aspects of the disclosure relate to a human cell comprising: i) an exogenous expression or activity inhibitor of, or ii) a genomic mutation of: one or more of β₂ microglobin (B2M), CIITA, TRAC, TRBC1, or TRBC2. In some embodiments, the cell comprises a genomic mutation. In some embodiments, the genomic mutation comprises a mutation of one or more endogenous genes in the cell's genome, wherein the one or more endogenous genes comprise the B2M, CIITA, TRAC, TRBC1, or TRBC2 gene. In some embodiments, the mutation comprises a loss of function mutation. In some embodiments, the inhibitor is an expression inhibitor. In some embodiments, the inhibitor comprises an inhibitory nucleic acid. In some embodiments, the inhibitory nucleic acid comprises one or more of a siRNA, shRNA, miRNA, or an antisense molecule. In some embodiments, the cells comprise an activity inhibitor. In some embodiments, following modification the cell is deficient in any detectable expression of one or more of B2M, CIITA, TRAC, TRBC1, or TRBC2 proteins. In some embodiments, the cell comprises an inhibitor or genomic mutation of B2M. In some embodiments, the cell comprises an inhibitor or genomic mutation of CIITA. In some embodiments, the cell comprises an inhibitor or genomic mutation of TRAC. In some embodiments, the cell comprises an inhibitor or genomic mutation of TRBC1. In some embodiments, the cell comprises an inhibitor or genomic mutation of TRBC2. In some embodiments, at least 90% of the genomic DNA encoding B2M, CIITA, TRAC, TRBC1, and/or TRBC2 is deleted. In some embodiments, at least or at most 5, 10, 20, 30, 40, 50, 60, 70, 80, 90, 95, 99, or 100% (or any range derivable therein) of the genomic DNA encoding B2M, CIITA, TRAC, TRBC1, and/or TRBC2 is deleted. In other embodiments, a deletion, insertion, and/or substitution is made in the genomic DNA. In some embodiments, the cell is a progeny of the human stem or progenitor cell.

The ^{U}HSC-γδ T cells that are modified to be HLA-negative may be genetically modified by any suitable manner. The genetic mutations of the disclosure, such as those in the CIITA and/or B2M genes can be introduced by methods known in the art. In certain embodiments, engineered nucleases may be used to introduce exogenous nucleic acid sequences for genetic modification of any cells referred to herein. Genome editing, or genome editing with engineered nucleases (GEEN) is a type of genetic engineering in which DNA is inserted, replaced, or removed from a genome using artificially engineered nucleases, or “molecular scissors.” The nucleases create specific double-stranded break (DSBs) at desired locations in the genome and harness the cell's endogenous mechanisms to repair the induced break by natural processes of homologous recombination (HR) and nonhomologous end-joining (NHEJ). Non-limiting engineered nucleases include Zinc finger nucleases (ZFNs), Transcription Activator-Like Effector Nucleases (TALENs), the CRISPR/Cas9 system, and engineered meganuclease re-engineered homing endonucleases. Any of the engineered nucleases known in the art can be used in certain aspects of the methods and compositions.

In cases wherein the engineered γδ T cells comprise one or more suicide genes for subsequent depletion upon need, the suicide gene may be of any suitable kind. The γδ T cells of the disclosure may express a suicide gene product that may be enzyme-based, for example. Examples of suicide gene products include herpes simplex virus thymidine kinase (HSV-TK), purine nucleoside phosphorylase (PNP), cytosine deaminase (CD), carboxypetidase G2, cytochrome P450, linamarase, beta-lactamase, nitroreductase (NTR), carboxypeptidase A, or inducible caspase 9. Thus, in specific cases, the suicide gene may encode thymidine kinase (TK). In specific cases, the TK gene is a viral TK gene, such as a herpes simplex virus TK gene. In particular embodiments, the suicide gene product is activated by a substrate, such as ganciclovir, penciclovir, or a derivative thereof.

In some embodiments, the engineered γδ T cells are able to be imaged or otherwise detected. In particular cases, the cells comprise an exogenous nucleic acid encoding a polypeptide that has a substrate that may be labeled for imaging, and the imaging may be fluorescent, radioactive, colorimetric, and so forth. In specific cases, the cells are detected by positron emission tomography. The cells in at least some cases express sr39TK gene that is a positron emission tomography (PET) reporter/thymidine kinase gene that allows for tracking of these genetically modified cells with PET imaging and elimination of these cells through the sr39TK suicide gene function.

Encompassed by the disclosure are populations of engineered γδ T cells. In particular aspects, γδ T clonal cells comprise an exogenous nucleic acid encoding an γδ T-cell receptor and lack surface expression of one or more HLA-I or HLA-II molecules. The γδ T cells may comprise an exogenous nucleic acid encoding a suicide gene, including an enzyme-based suicide gene such as thymidine kinase (TK). The TK gene may be a viral TK gene, such as a herpes simplex virus TK gene. In the cells of the population the suicide gene may be activated by a substrate, such as ganciclovir, penciclovir, or a derivative thereof, for example. The cells may comprise an exogenous nucleic acid encoding a polypeptide that has a substrate that may be labeled for imaging, and in some cases a suicide gene product is the polypeptide that has a substrate that may be labeled for imaging. In specific aspects, the suicide gene is sr39TK. In particular cases for the γδ T cell population, the γδ T cells comprise nucleic acid sequences from a recombinant vector that was introduced into the cells, such as a viral vector (including at least a lentivirus, a retrovirus, an adeno-associated virus (AAV), a herpesvirus, or adenovirus).

In certain embodiments, the cells of the γδ T cell population may or may not have been exposed to, or are exposed to, one or more certain conditions. In certain cases, for example, the cells of the population not exposed or were not exposed to media that comprises animal serum. The cells of the population may or may not be frozen. In some cases, the cells of the population are in a solution comprising dextrose, one or more electrolytes, albumin, dextran, and/or DMSO. The solution may comprise dextrose, one or more electrolytes, albumin, dextran, and DMSO. The cells may be in a solution that is sterile, nonpyogenic, and isotonic. In specific cases the γδ T cells have been activated, such as activated with ZOL. In specific aspects, the cell population comprises at least about 10²-10⁶ clonal cells. The cell population may comprise at least about 10⁶-10¹² total cells, in some cases.

In particular embodiments there is an gamma delta (γδ) T cell population comprising: clonal γδ T cells comprising one or more exogenous nucleic acids encoding an γδ T-cell receptor and a thymidine kinase suicide, wherein the clonal γδ T cells have been engineered not to express functional beta-2-microglobulin (B2M), major histocompatibility complex class II transactivator (CIITA), and/or HLA-I and HLA-II molecules and wherein the cell population is at least about 10⁶-10¹² total cells and comprises at least about 10²-10⁶ clonal cells. In some cases, the cells are frozen in a solution.

In particular embodiments, the ^{U}HSC-γδ T cells and/or precursors thereto may be specifically formulated and/or they may be cultured in a particular medium (whether or not they are present in an in vitro ATO culture system) at any stage of a process of generating the ^{U}HSC-γδ T cells. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In further embodiments, the medium may be a serum-free medium that is suitable for cell development. For example, the medium may comprise B-27© supplement, xeno-free B-27© supplement (available at world wide web at thermofisher.com/us/en/home/technical-resources/media-formulation.250.html), NS21 supplement (Chen et al., J Neurosci Methods, 2008 Jun. 30; 171(2): 239-247, incorporated herein in its entirety), GS21™ supplement (available at world wide web at amsbio.com/B-27.aspx), or a combination thereof at a concentration effective for producing T cells from the 3D cell aggregate.

Cell expressing polypeptides comprising an amino acid sequence shown in Table 1 (SEQ ID NO: 1-SEQ ID NO: 52) and/or other γδ T cells may be produced by any suitable method(s). The method(s) may utilize one or more successive steps for one or more modifications to cells and/or utilize one or more simultaneous steps for one or more modifications to cells. In specific embodiments, a starting source of cells are modified to become functional as γδ T cells followed by one or more steps to add one or more additional characteristics to the cells, such as the ability to be imaged, and/or the ability to be selectively killed, and/or the ability to be able to be used allogeneically. In specific embodiments, at least part of the process for generating ^{U}HSC-γδ T cells occurs in a specific in vitro culture system. An example of a specific in vitro culture system is one that allows differentiation of certain cells at high efficiency and high yield. In specific embodiments the in vitro culture system is an artificial thymic organoid (ATO) system.

In specific cases, ^{U}HSC-γδ T cells may be generated by the following: 1) genetic modification of donor HSCs to express γδ TCRs (for example, via lentiviral vectors) and to eliminate expression of HLA-I/II molecules (for example, via CRISPR/Cas9-based gene editing); 2) in vitro differentiation into γδ T cells via an ATO culture, 3) in vitro γδ T cell purification and expansion, and 4) formulation and cryopreservation and/or use.

Particular embodiments of the disclosure provide methods of preparing a population of clonal gamma delta (T6) T cells comprising: a) selecting CD34⁺ cells from human peripheral blood cells (PBMCs); b) introducing one or more nucleic acids encoding a human γδ T-cell receptor (TCR); c) eliminating expression of one or more HLA-I/II genes in the isolated human CD34⁺ cells; and, d) culturing isolated CD34⁺ cells expressing γδ TCR in an artificial thymic organoid (ATO) system to produce γδ T cells, wherein the ATO system comprises a 3D cell aggregate comprising a selected population of stromal cells that express a Notch ligand and a serum-free medium. The method may further comprise isolating CD34⁻ cells. In alternative embodiments, other culture systems than the ATO system is employed, such as a 2-D culture system or other forms of 3-D culture systems (e.g., FTOC-like culture, metrigel-aided culture).

Specific aspects of the disclosure relate to a novel three-dimensional cell culture system to produce γδ T cells from less differentiated cells such as embryonic stem cells, pluripotent stem cells, hematopoietic stem or progenitor cells, induced pluripotent stem (iPS) cells, or stem or progenitor cells. Stem cells of any type may be utilized from various resources, including at least fetal liver, cord blood, and peripheral blood CD34⁺ cells (either G-CSF-mobilized or non-G-CSF-mobilized), for example.

In particular embodiments, the system involves using serum-free medium. In certain aspects, the system uses a serum-free medium that is suitable for cell development for culturing of a three-dimensional cell aggregate. Such a system produces sufficient amounts of ^{U}HSC-γδ T cells. In embodiments of the disclosure, the 3D cell aggregate is cultured in a serum-free medium comprising insulin for a time period sufficient for the in vitro differentiation of stem or progenitor cells to ^{U}HSC-γδ T cells or precursors to ^{U}HSC-γδ T cells.

Embodiments of a cell culture composition comprise an ATO 3D culture that uses highly-standardized, serum-free components and a stromal cell line to facilitate robust and highly reproducible T cell differentiation from human HSCs. In certain embodiments, cell differentiation in ATOs closely mimicked endogenous thymopoiesis and, in contrast to monolayer co-cultures, supported efficient positive selection of functional ^{U}HSC-γδ T. Certain aspects of the 3D culture compositions use serum-free conditions, avoid the use of human thymic tissue or proprietary scaffold materials, and facilitate positive selection and robust generation of fully functional, mature human ^{U}HSC-γδ T cells from source cells.

Cells produced by the preparation methods may be frozen. The produced cells may be in a solution comprising dextrose, one or more electrolytes, albumin, dextran, and DMSO. The solution may be sterile, nonpyogenic, and isotonic.

Genetic modification may also be introduced to certain components to generate antigen-specific T cells, and to model positive and negative selection. Examples of these modifications include transduction of HSCs with a lentiviral vector encoding an antigen-specific T cell receptor (TCR) or chimeric antigen receptor (CAR) for the generation of antigen-specific, allelically excluded naïve T cells; transduction of HSCs with gene/s to direct lineage commitment to specialized lymphoid cells. For example, transduction of HSCs with a gamma delta (76) associated TCR to generate functional γδ T cells in ATOs; transduction of the ATO stromal cell line (e.g., MS5-hDLL1) with human MHC genes (e.g. human CD1d gene) to enhance positive selection and maturation of both TCR engineered or non-engineered T cells in ATOs; and/or transduction of the ATO stromal cell line with an antigen plus costimulatory molecules or cytokines to enhance the positive selection of CAR T cells in ATOs.

In producing the engineered γδ T cells, CD34⁺ cells from human peripheral blood cells (PBMCs) may be modified by introducing certain exogenous gene(s) and by knocking out certain endogenous gene(s). The methods may further comprise culturing selected CD34⁺ cells in media prior to introducing one or more nucleic acids into the cells. The culturing may comprise incubating the selected CD34⁺ cells with medium comprising one or more growth factors, in some cases, and the one or more growth factors may comprise c-kit ligand, flt-3 ligand, and/or human thrombopoietin (TPO), for example. The growth factors may or may not be at a certain concentration, such as between about 5 ng/ml to about 500 ng/ml.

In particular methods the nucleic acid(s) to be introduced into cells are one or more nucleic acids that comprise a nucleic acid sequence encoding an γ-TCR and a S-TCR (e.g., SEQ ID NO: 1-SEQ ID NO: 52). The methods may comprise introducing into the selected cells a nucleic acid encoding a suicide gene. In specific aspects, one nucleic acid encodes both the γ-TCR and the S-TCR, or one nucleic acid encodes the γ-TCR, the S-TCR, and the suicide gene. The suicide gene may be enzyme-based, such as thymidine kinase (TK) including a viral TK gene such as one from herpes simplex virus TK gene. The suicide gene may be activated by a substrate, such as ganciclovir, penciclovir, or a derivative thereof. The cells may be engineered to comprise an exogenous nucleic acid encoding a polypeptide that has a substrate that may be labeled for imaging. In some cases, a suicide gene product is a polypeptide that has a substrate that may be labeled for imaging, such as sr39TK.

In manufacturing the engineered γδ T cells, the cells may be present in a particular serum-free medium, including one that comprises externally added ascorbic acid. In specific aspects, the serum-free medium further comprises externally added FLT3 ligand (FLT3L), interleukin 7 (IL-7), stem cell factor (SCF), thrombopoietin (TPO), stem cell factor (SCF), thrombopoietin (TPO), IL-2, IL-4, IL-6, IL-15, IL-21, TNF-alpha, TGF-beta, interferon-gamma, interferon-lambda, TSLP, thymopentin, pleotrophin, midkine, or combinations thereof. The serum-free medium may further comprise vitamins, including biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or combinations thereof or salts thereof. The serum-free medium may further comprise one or more externally added (or not) proteins, such as albumin or bovine serum albumin, a fraction of BSA, catalase, insulin, transferrin, superoxide dismutase, or combinations thereof. The serum-free medium may further comprise corticosterone, D-Galactose, ethanolamine, glutathione, L-carnitine, linoleic acid, linolenic acid, progesterone, putrescine, sodium selenite, or triodo-I-thyronine, or combinations thereof. The serum-free medium may comprise a B-27© supplement, xeno-free B-27© supplement, GS21™ supplement, or combinations thereof. Amino acids (including arginine, cysteine, isoleucine, leucine, lysine, methionine, glutamine, phenylalanine, threonine, tryptophan, histidine, tyrosine, or valine, or combinations thereof), monosaccharides, and/or inorganic ions (including sodium, potassium, calcium, magnesium, nitrogen, or phosphorus, or combinations or salts thereof, for example) may be present in the serum-free medium. The serum-free medium may further comprise molybdenum, vanadium, iron, zinc, selenium, copper, or manganese, or combinations thereof.

Further aspects and embodiments of the invention are discussed in the following sections.

### EXAMPLES

### Human Vγ9Vδ2 TCR Clones, Sequences, and Gene Delivery Vectors

Human Vγ9Vδ2 TCRs (referred to as γδ TCRs herein) were cloned from healthy donor peripheral blood mononuclear cells (PBMCs)-derived γδ T (PBMC-γδT) cells. Illustrative working embodiments of the methods disclosed herein as well as γδ TCR sequences (e.g., amino acid sequences and/or gene coding sequences) and illustrative γδ TCR gene delivery vectors are discussed below.

### Methods

Human γδ T cells can be generated through γδ TCR gene-engineering of stem and progenitor cells (e.g., CD34⁺ HSCs, ESCs, iPSCs), followed by differentiation (in vivo or ex vivo) into transgenic γδ T cells.

HSCs refer to human CD34⁺ hematopoietic progenitor and stem cells, that can be directly isolated from cord blood or G-CSF-mobilized peripheral blood (CB HSCs or PBSCs), or can be derived from embryonic or induced pluripotent stem cells (ES-HSCs or iPS-HSCs). HSCs can be gene engineered via vector-dependent or vector-independent gene delivery methods, or via other gene editing methods (e.g., CRISPR, TALEN, Zinc finger and the like.

In addition to the antigen-specificity endowed by the monoclonal transgenic γδ TCR, HSC-γδT can be further engineered to express additional targeting molecules to enhance their disease-targeting capacity. Such targeting molecules can be Chimeric Antigen Receptors (CARs), natural or synthetic receptors/ligands, or others. The resulting CAR-γδT cells can then be utilized for off-the-shelf disease-targeting cellular therapy.

In addition to the antigen-specificity endowed by the monoclonal transgenic TCR, HSC-γδT can be further engineered to express additional targeting molecules to enhance their disease-targeting capacity. Such targeting molecules can be Chimeric Antigen Receptors (CARs), natural or synthetic receptors/ligands, or others. The resulting CAR-γδT cells can then be utilized for off-the-shelf disease-targeting cellular therapy.

The HSC-γδT cells and derivatives can also be further engineered to overexpress genes encoding T cell stimulatory factors, or to disrupt genes encoding T cell inhibitory factors, resulting in functionally enhanced HSC-γδT cells and derivatives.

### In Vivo Generation of HSC-Engineered γδT (HSC-γδT) Cells for HSC Adoptive Therapy

A γδ TCR gene-engineered HSC adoptive transfer method is disclosed that can generate HSC-γδT cells in vivo, cells that can potentially provide patients with a life-long supply of engineered HSC-γδT cells targeting diseases.

The procedure includes 1) genetic modification of human CD34⁺ hematopoietic stem cells (HSCs) to express a selected γδ TCR gene; 2) adoptive transfer γδ TCR gene engineered HSCs into a patient; 3) in vivo generation of HSC-γδT cells; 4) due to longevity of self-renewal of HSCs, this method can potentially protect patient with life-long supplies of HSC-γδT cells.

### Ex Vivo Generation of Allogenic HSC-Engineered γδ T (^{Allo}HSC-γδT) Cells for Off-the-Shelf Cell Therapy

Ex vivo differentiation culture methods are disclosed to generate ^{Allo}HSC-γδT cells for off-the-shelf cell therapy applications.

### Feeder-Dependent Cultures

The procedure includes 1) genetic modification of human CD34⁺ hematopoietic stem cells (HSCs) to express a selected γδ TCR gene; 3) ex vivo generation of ^{Allo}HSC-γδT cells with feeder cells (e.g., artificial thymic organoid culture; 3) ex vivo expansion of differentiated ^{Allo}HSC-γδT cells.

### Feeder-Free Cultures

The production procedure includes 1) genetic modification of human CD34⁺ hematopoietic stem cells (HSCs) to express a selected TCR gene; 2) ex vivo differentiation ^{Allo}HSC-γδT cells without feeder cells; and 3) ex vivo expansion of differentiated ^{Allo}HSC-γδT cells.

### Applications

Engineered γδ T cells can be used to target multiple diseases including cancer and infectious diseases.

### γδ T Cell Therapy for Cancer

Proof of principle data are provided for treating a large collection of human cancers, including blood cancer (e.g., multiple myeloma) and solid tumor (e.g., ovarian, melanoma, prostate, breast, and lung cancer).

### γδ T Cell Therapy for Infectious Diseases

Proof of principle data are provided for targeting COVID-19.

### Detailed Description of the ^{Allo}HSC-γδT Cell Culture Methods

### In Vivo Generation of HSC-γδT Cells

Human CD34⁺ HSCs were cultured for no more than 48 hours in X-VIVO 15 serum-free hematopoietic cell medium containing recombinant human Flt3 ligand, SCF, TPO, and IL-3 in no-tissue culture-treated plates coated with Retronectin. Viral transduction was performed at 24 hours by adding concentrated lentivector directly to the culture medium. At around 48 hours CD34⁺ cells were collected and intravenously (i.v.) injected in NOD.Cg-Prkd^{scid} Il2rg^{tm1/Wji}/SzJ (NSG) mice that had received 270 rads of total body irradiation. 1-2 fragments of human fetal or postnatal thymus were implanted under the kidney capsule of each recipient NSG mice.

### Feeder-Dependent Ex Vivo Generation of ^{Allo}HSC-γδ T Cells

### Stage 1: ^{Allo}HSC-γδT Cell Differentiation

Fresh or frozen/thawed CD34⁺ HSCs are cultured in stem cell culture media (base medium supplemented with cytokine cocktails including IL-3, IL-7, IL-6, SCF, EPO, TPO, FLT3L, and others) for 12-72 hours in flasks coated with retronectin, followed by addition of the TCR gene-delivery vector, and culturing for an additional 12-48 hours. TCR gene-modified HSCs are then differentiated into ^{Allo}HSC-γδT cells in a feeder-dependent culture (e.g., artificial thymic organoid culture) over 4-10 weeks. Artificial thymic organoid (ATO) was generated following a previously established protocol (Seet et al., Cell Stem Cell. 2019 Mar. 7; 24(3):376-389).

### Stage 2: ^{Allo}HSC-γδT Cell Expansion

At Stage 2, differentiated ^{Allo}HSC-γδT cells are stimulated with TCR cognate antigens (proteins, peptides, lipids, phosphor-antigens, small molecules, and others) or non-specific TCR stimulatory reagents (anti-CD3/anti-CD28 antibodies or antibody-coated beads, Concanavalin A, PMA/Ionomycin, and others), and expanded for up to 1 month in T cell culture media. The culture can be supplemented with T cell supporting cytokines (IL-2, IL-7, IL-15, and others). ^{Allo}HSC-γδT Cell Derivatives

In some embodiments, ^{Allo}HSC-γδT cells can be further engineered to express additional transgenes. In one embodiment, such transgenes encode disease targeting molecules such as chimeric antigen receptors (CARs), T-cell receptors (TCRs), and other native or synthetic receptor/ligands. In another embodiment, such transgenes can encode T cell regulatory proteins such as IL-2, IL-7, IL-15, IFN-γ, TNF-α, CD28, 4-1BB, OX40, ICOS, FOXP3, and others. Transgenes can be introduced into post-expansion ^{Allo}HSC-γδT cells or their progenitor cells (HSCs, newly differentiated ^{Allo}HSC-γδT cells, in-expansion ^{Allo}HSC-γδT cells) at various culture stages.

In some embodiments, ^{Allo}HSC-γδT cells can be further engineered to disrupt selected genes using gene editing tools (CRISPR, TALEN, Zinc-Finger, and others). In one embodiment, disrupted genes encode T cell immune checkpoint inhibitors (PD-1, CTLA-4, TIM-3, LAG-3, and others). Deficiency of these negative regulatory genes may enhance the disease fighting capacity of ^{Allo}HSC-γδT cells, making them resistance to disease-induced anergy and tolerance.

### Feeder-Free Ex Vivo Generation of ^{Allo}HSC-γδT Cells

### Stage 1: ^{Allo}HSC-γδT Cell Differentiation

Fresh or frozen/thawed CD34⁺ HSCs are cultured in stem cell culture media (base medium supplemented with cytokine cocktails including IL-3, IL-7, IL-6, SCF, EPO, TPO, FLT3L, and others) for 12-72 hours in flasks coated with retronectin, followed by addition of the TCR gene-delivery vector, and culturing for an additional 12-48 hours.

TCR gene-modified HSCs are then differentiated into ^{Allo}HSC-γδT cells in a differentiation medium over a period of 4-10 weeks without feeders. Non-tissue culture-treated plates are coated with a ^{Allo}HSC-γδT Culture Coating Material (DLL-1/4, VCAM-1/5, retronectin, and others). CD34⁺ HSCs are suspended in an Expansion Medium (base medium containing serum albumin, recombinant human insulin, human transferrin, 2-mercaptoethanol, SCF, TPO, IL-3, IL-6, Flt3 ligand, human LDL, UM171, and additives), seeded into the coated wells of a plate, and cultured for 3-7 days. Expansion Medium is refreshed every 3-4 days. Cells are then collected and suspended in a Maturation Medium (base medium containing serum albumin, recombinant human insulin, human transferrin, 2-mercaptoethanol, SCF, IL-3, IL-6, IL-7, IL-15, Flt3 ligand, ascorbic acid, and additives). Maturation Medium is refreshed 1-2 times per week.

### Stage 2: ^{Allo}HSC-γδT Cell Expansion

Differentiated ^{Allo}HSC-γδT cells are stimulated with TCR cognate antigens (proteins, peptides, lipids, phosphor-antigens, small molecules, and others) or non-specific TCR stimulatory reagents (anti-CD3/anti-CD28 antibodies or antibody-coated beads, Concanavalin A, PMA/Ionomycin, and artificial APCs), and expanded for up to 1 month in T cell culture media. The culture can be supplemented with T cell supporting cytokines (IL-2, IL-7, IL-15, and others).

### ^{Allo}HSC-γδT Cell Derivatives

In some embodiments, ^{Allo}HSC-γδT cells can be further engineered to express additional transgenes. In one embodiment, such transgenes encode disease targeting molecules such as chimeric antigen receptors (CARs), T-cell receptors (TCRs), and other native or synthetic receptor/ligands. In another embodiment, such transgenes can encode T cell regulatory proteins such as IL-2, IL-7, IL-15, IFN-γ, TNF-α, CD28, 4-1BB, OX40, ICOS, FOXP3, and others. Transgenes can be introduced into post-expansion ^{Allo}HSC-γδT cells or their progenitor cells (HSCs, newly differentiated ^{Allo}HSC-γδT cells, in-expansion ^{Allo}HSC-γδT cells) at various culture stages.

In some embodiments, ^{Allo}HSC-γδT cells can be further engineered to disrupt selected genes using gene editing tools (CRISPR, TALEN, Zinc-Finger, and others). In one embodiment, disrupted genes encode T cell immune checkpoint inhibitors (PD-1, CTLA-4, TIM-3, LAG-3, and others). Deficiency of these negative regulatory genes may enhance the disease fighting capacity of ^{Allo}HSC-γδT cells, making them resistance to disease-induced anergy and tolerance.

In some embodiments, ^{Allo}HSC-γδT cells or enhanced ^{Allo}HSC-γδT cells can be further engineered to make them suitable for allogeneic adoptive transfer, thereby suitable for serving as off-the-shelf cellular products. In one embodiment, genes encoding MHC molecules or MHC expression/display regulatory molecules [MHC molecules, B2M, CIITA (Class II transcription activator control induction of MHC class II mRNA expression), and others]. Lack of MHC molecule expression on ^{Allo}HSC-γδT cells makes them resistant to allogeneic host T cell-mediated depletion. In another embodiment, MHC class-I deficient ^{Allo}HSC-γδT cells will be further engineered to overexpress an HLA-E gene that will endow them resistant to host NK cell-mediated depletion.

^{Allo}HSC-γδT cells and derivatives can be used freshly or cryopreserved for further usage. Moreover, various intermediate cellular products generated during ^{Allo}HSC-γδT cell culture can be paused for cryopreservation, stored and recovered for continued production.

### Novel Features and Advantages

Compared to the method of generating ^{Allo}HSC-γδT cells using a feeder-dependent culture (e.g., ATO culture), this invention offers an in vitro differentiation method that does not require feeder cells. This new method greatly improves the process for the scale-up production and GMP-compatible manufacturing of therapeutic cells for human applications.

The cell products, ^{Allo}HSC-γδT cells, display phenotypes/functionalities distinct from that of their native counterpart T cells as well as their counterpart T cells generated using other ex vivo culture methods (e.g., ATO culture method), making ^{Allo}HSC-γδT cells unique cellular products.

Unique features of the ^{Allo}HSC-γδT cell differentiation culture include:

1) It is Ex Vivo and Feeder-Free.

2) It does not support TCR V/D/J recombination, so no randomly rearranged endogenous TCRs, thereby no GvHD risk.

3) It supports the synchronized differentiation of transgenic ^{Allo}HSC-γδT cells, thereby eliminating the presence of un-differentiated progenitor cells and other lineages of bystander immune cells.

4) As a result, the ^{Allo}HSC-γδT cell product comprises a homogenous and pure population of monoclonal TCR engineered T cells. No escaped random T cells, no other lineages of immune cells, and no un-differentiated progenitor cells. Therefore, no need for a purification step.

5) High yield. About 10^{13 Allo}HSC-γδT cells (10,000-100,000 doses) can be generated from PBSCs of a healthy donor, and about 10^{13 Allo}HSC-γδT cells (10,000-100,000 doses) can be generated from CB HSCs of a healthy donor.

6) Unique phenotype of ^{Allo}HSC-γδT cells—transgenicTCR⁺endogenousTCR⁻CD3⁺.

(Note: These unique features of the ^{Allo}HSC-γδT cell differentiation culture distinct it from other methods to generate off-the-shelf T cell products, including the healthy donor PBMC-based T cell culture, the ATO culture, and the others.)

### Proof of Principle

Proof-of-principle studies have been performed, showing the successful generation of ^{Allo}HSC-γδT cells. Further engineering of ^{Allo}CAR-γδT cells to additionally express a BCMA CAR (^{Allo}BCAR-γδT cell product) and together with Interleukin-15 (IL-15) (^{Allo15}BCAR-γδT cell product) were also proved successful. Pilot CMC, pharmacology, efficacy, and safety studies were performed analyzing these cell products.

TABLE 1
AMINO ACID SEQUENCES OF CLONED γδ TCR CDR3 REGIONS
Human γδ TCR genes were cloned using a single-cell RT-PCR approach (see, e.g., FIG. 1).
Briefly, human γδ T cells were expanded from healthy donor peripheral blood mononuclear cells
(PBMCs) and sorted using flow cytometry based on a stringent combination of surface markers,
gated as hCD3+Vγ9+Vδ2+ (FIGS. 1A and 1B). Single cells were sorted directly into PCR plates
containing cell lysis buffer and then subjected to TCR cloning using a one-step RT-PCR followed
by Sanger sequencing analysis (FIG. 1A). As shown below, over 25 pairs of γδ TCR γ9 and δ2
chain genes were identified.

| Label | γ9-CDR3 | δ2-CDR3 |
|---|---|---|
| G115* | ALVVEAQQELGKKIKVFGPGTKLIIT | ACDTLGMGGEYTDKLIFGKGTRVTVEP |
| | (SEQ ID NO.: 1) | (SEQ ID NO.: 2) |
| LYγδ1 | ALWEVRELGKKIKVFGPGTKLIIT | ACDTVGGATDKLIFGKGTRVTVEP |
| | (SEQ ID NO.: 3) | (SEQ ID NO.: 4) |
| LYγδ2 | ALWEPQELGKKIKVFGPGTKLIIT | ACDPLLGDRYTDKLIFGKGTRVTVEP |
| | (SEQ ID NO.: 5) | (SEQ ID NO.: 6) |
| LYγδ3 | ALWEVQELGKKIKVFGPGTKLIIT | ACDNGDTRSVVDTRQMFFGTGIKLFVEP |
| | (SEQ ID NO.: 7) | (SEQ ID NO.: 8) |
| LYγδ4 | ALVVEDQELGKKIKVFGPGTKLIIT | ACDPWGTLDKLIFGKGTRVTVEP |
| | (SEQ ID NO.: 9) | (SEQ ID NO.: 10) |
| LYγδ5 | ALVVDQQELGKKIKVFGPGTKLIIT | ACAAAGGSVVDTRQMFFGTGIKLFVEP |
| | (SEQ ID NO.: 11) | (SEQ ID NO.: 12) |
| LYγδ6 | ALWEVKELGKKIKVFGPGTKLIIT | ACDTVMYTDKLIFGKGTRVTVEP |
| | (SEQ ID NO.: 13) | (SEQ ID NO.: 14) |
| LYγδ7 | ALWEVEELGKKIKVFGPGTKLIIT | ALSPLGLGDTDKLIFGKGTRVTVEP |
| | (SEQ ID NO.: 15) | (SEQ ID NO.: 16) |
| LYγδ8 | ALWEFQELGKKIKVFGPGTKLIIT | ACDKVSRTGGSQYTDKLIFGKGTRVTVEP |
| | (SEQ ID NO.: 17) | (SEQ ID NO.: 18) |
| LYγδ9 | ALVVDQSQELGKKIKVFGPGTKLIIT | ACDTLLGDTRRSSSVVDTRQMFFGTGIKLFVEP |
| | (SEQ ID NO.: 19) | (SEQ ID NO.: 20) |
| LYγδ10 | ALVVEVLELGKKIKVFGPGTKLIIT | ACDTVSTFRGGPDKLIFGKGTRVTVEP |
| | (SEQ ID NO.: 21) | (SEQ ID NO.: 22) |
| LYγδ11 | ALTGQELGKKIKVFGPGTKLIIT | ACDKVVGGGYAADTDKLIFGKGTRVTVEP |
| | (SEQ ID NO.: 23) | (SEQ ID NO.: 24) |
| LYγδ12 | ALWEVSELGKKIKVFGPGTKLIIT | ACDTVVVGLGLGDKLIFGKGTRVTVEP |
| | (SEQ ID NO.: 25) | (SEQ ID NO.: 26) |
| LYγδ13 | ALVVEANQELGKKIKVFGPGTKLIIT | ACDKLGDTREKLIFGKGTRVTVEP |
| | (SEQ ID NO.: 27) | (SEQ ID NO.: 28) |
| LYγδ14 | ALWEVKLGKKIKVFGPGTKLIIT | ACAPLGDRGSWDTRQMFFGTGIKLFVEP |
| | (SEQ ID NO.: 29) | (SEQ ID NO.: 30) |
| LYγδ15 | ALWEASELGKKIKVFGPGTKLIIT | ACEPLRTGGPKVDKLIFGKGTRVTVEP |
| | (SEQ ID NO.: 31) | (SEQ ID NO.: 32) |
| LYγδ16 | ALWEAQELGKKIKVFGPGTKLIIT | ACDSGGYSSVVDTRQMFFGTGIKLFVEP |
| | (SEQ ID NO.: 33) | (SEQ ID NO.: 34) |
| LYγδ17 | ALWEVQELGKKIKVFGPGTKLIIT | ACDRLLGDTDKLIFGKGTRVTVEP |
| | (SEQ ID NO.: 35) | (SEQ ID NO.: 36) |
| LYγδ18 | ALVVEAHQELGKKIKVFGPGTKLIIT | ACDSLGDSVDKLIFGKGTRVTVEP |
| | (SEQ ID NO.: 37) | (SEQ ID NO.: 38) |
| LYγδ19 | ALVVEDLELGKKIKVFGPGTKLIIT | ACDTVSWGKNTDKLIFGKGTRVTVEP |
| | (SEQ ID NO.: 39) | (SEQ ID NO.: 40) |
| LYγδ20 | ALWEVRELGKKIKVFGPGTKLIIT | ACDTIVSGYDGYDKLIFGKGTRVTVEP |
| | (SEQ ID NO.: 41) | (SEQ ID NO.: 42) |
| LYγδ21 | ALVVVQELGKKIKVFGPGTKLIIT | ACDVLGDTEADKLIFGKGTRVTVEP |
| | (SEQ ID NO.: 43) | (SEQ ID NO.: 44) |
| LYγδ22 | ALVVEVRQELGKKIKVFGPGTKLIIT | ACDTVSQRGGYSDKLIFGKGTRVTVEP |
| | (SEQ ID NO.: 45) | (SEQ ID NO.: 46) |
| LYγδ23 | ALWESKELGKKIKVFGPGTKLIIT | ACEGLGATQSSWDTRQMFFGTGIKLFVEP |
| | (SEQ ID NO.: 47) | (SEQ ID NO.: 48) |
| LYγδ24 | ALWGGELGKKIKVFGPGTKLIIT | ACDLLGDTRYTDKLIFGKGTRVTVEP |
| | (SEQ ID NO.: 49) | (SEQ ID NO.: 50) |
| LYγδ25 | ALWDIPPGQELGKKIKVFGPGTKLIIT | ACDTLGETSSWDTRQMFFGTGIKLFVEP |
| | (SEQ ID NO.: 51) | (SEQ ID NO.: 52) |

*G115 is a previously reported clone of Vγ9Vδ2 TCR (Allison 2001, Nature 411:820).

### ILLUSTRATIVE VECTOR SEQUENCES

pMNDW-G115 DNA sequence:
TCRγ9(G115 CDR3)-T2A-TCRδ2(G115 CDR3)
(SEQ ID NO: 53)
CAGGTGGCACTTTTCGGGGAAATGTGCGCGGAACCCCTATTTGTTTATTTTTCTAAAT
ACATTCAAATATGTATCCGCTCATGAGACAATAACCCTGATAAATGCTTCAATAATA
TTGAAAAAGGAAGAGTATGAGTATTCAACATTTCCGTGTCGCCCTTATTCCCTTTTTT
GCGGCATTTTGCCTTCCTGTTTTTGCTCACCCAGAAACGCTGGTGAAAGTAAAAGAT
GCTGAAGATCAGTTGGGTGCACGAGTGGGTTACATCGAACTGGATCTCAACAGCGG
TAAGATCCTTGAGAGTTTTCGCCCCGAAGAACGTTTTCCAATGATGAGCACTTTTAA
AGTTCTGCTATGTGGCGCGGTATTATCCCGTATTGACGCCGGGCAAGAGCAACTCGG
TCGCCGCATACACTATTCTCAGAATGACTTGGTTGAGTACTCACCAGTCACAGAAAA
GCATCTTACGGATGGCATGACAGTAAGAGAATTATGCAGTGCTGCCATAACCATGA
GTGATAACACTGCGGCCAACTTACTTCTGACAACGATCGGAGGACCGAAGGAGCTA
ACCGCTTTTTTGCACAACATGGGGGATCATGTAACTCGCCTTGATCGTTGGGAACCG
GAGCTGAATGAAGCCATACCAAACGACGAGCGTGACACCACGATGCCTGTAGCAAT
GGCAACAACGTTGCGCAAACTATTAACTGGCGAACTACTTACTCTAGCTTCCCGGCA
ACAATTAATAGACTGGATGGAGGCGGATAAAGTTGCAGGACCACTTCTGCGCTCGG
CCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCT
ACACGACGGGGAGTCAGGCAACTATGGATGAACGAAATAGACAGATCGCTGAGATA
GGTGCCTCACTGATTAAGCATTGGTAACTGTCAGACCAAGTTTACTCATATATACTTT
AGATTGATTTAAAACTTCATTTTTAATTTAAAAGGATCTAGGTGAAGATCCTTTTTGA
TAATCTCATGACCAAAATCCCTTAACGTGAGTTTTCGTTCCACTGAGCGTCAGACCC
CGTAGAAAAGATCAAAGGATCTTCTTGAGATCCTTTTTTTCTGCGCGTAATCTGCTGC
TTGCAAACAAAAAAACCACCGCTACCAGCGGTGGTTTGTTTGCCGGATCAAGAGCT
ACCAACTCTTTTTCCGAAGGTAACTGGCTTCAGCAGAGCGCAGATACCAAATACTGT
CCTTCTAGTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGTCGGGCT
GAACGGGGGGTTCGTGCACACAGCCCAGCTTGGAGCGAACGACCTACACCGAACTG
AGATACCTACAGCGTGAGCTATGAGAAAGCGCCACGCTTCCCGAAGGGAGAAAGGC
GGACAGGTATCCGGTAAGCGGCAGGGTCGGAACAGGAGAGCGCACGAGGGAGCTT
CCAGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCCAG
CAACGCGGCCTTTTTACGGTTCCTGGCCTTTTGCTGGCCTTTTGCTCACATGTTCTTTC
CTGCGTTATCCCCTGATTCTGTGGATAACCGTATTACCGCCTTTGAGTGAGCTGATAC
CGCTCGCCGCAGCCGAACGACCGAGCGCAGCGAGTCAGTGAGCGAGGAAGCGGAA
GAGCGCCCAATACGCAAACCGCCTCTCCCCGCGCGTTGGCCGATTCATTAATGCAGC
TGGCACGACAGGTTTCCCGACTGGAAAGCGGGCAGTGAGCGCAACGCAATTAATGT
GAGTTAGCTCACTCATTAGGCACCCCAGGCTTTACACTTTATGCTTCCGGCTCGTATG
TTGTGTGGAATTGTGAGCGGATAACAATTTCACACAGGAAACAGCTATGACCATGAT
TACGCCAAGCGCGCAATTAACCCTCACTAAAGGGAACAAAAGCTGGAGCTGCAAGC
TTGGCCATTGCATACGTTGTATCCATATCATAATATGTACATTTATATTGGCTCATGT
CCAACATTACCGCCATGTTGACATTGATTATTGACTAGTTATTAATAGTAATCAATTA
CGGGGTCATTAGTTCATAGCCCATATATGGAGTTCCGCGTTACATAACTTACGGTAA
ATGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGAGTATT
TACGGTAAACTGCCCACTTGGCAGTACATCAAGTGTATCATATGCCAAGTACGCCCC
CTATTGACGTCAATGACGGTAAATGGCCCGCCTGGCATTATGCCCAGTACATGACCT
TATGGGACTTTCCTACTTGGCAGTACATCTACGTATTAGTCATCGCTATTACCATGGT
GATGCGGTTTTGGCAGTACATCAATGGGCGTGGATAGCGGTTTGACTCACGGGGATT
TCCAAGTCTCCACCCCATTGACGTCAATGGGAGTTTGTTTTGGCACCAAAATCAACG
GGACTTTCCAAAATGTCGTAACAACTCCGCCCCATTGACGCAAATGGGCGGTAGGC
GTGTACGGTGGGAGGTCTATATAAGCAGAGCTCGTTTAGTGAACCGGGGTCTCTCTG
GTTAGACCAGATCTGAGCCTGGGAGCTCTCTGGCTAACTAGGGAACCCACTGCTTAA
GCCTCAATAAAGCTTGCCTTGAGTGCTTCAAGTAGTGTGTGCCCGTCTGTTGTGTGA
CTCTGGTAACTAGAGATCCCTCAGACCCTTTTAGTCAGTGTGGAAAATCTCTAGCAG
TGGCGCCCGAACAGGGACCTGAAAGCGAAAGGGAAACCAGAGGAGCTCTCTCGAC
GCAGGACTCGGCTTGCTGAAGCGCGCACGGCAAGAGGCGAGGGGCGGCGACTGGT
GAGTACGCCAAAAATTTTGACTAGCGGAGGCTAGAAGGAGAGAGATGGGTGCGAG
AGCGTCAGTATTAAGCGGGGGAGAATTAGATCGCGATGGGAAAAAATTCGGTTAAG
GCCAGGGGGAAAGAAAAAATATAAATTAAAACATATAGTATGGGCAAGCAGGGAG
CTAGAACGATTCGCAGTTAATCCTGGCCTGTTAGAAACATCAGAAGGCTGTAGACA
AATACTGGGACAGCTACAACCATCCCTTCAGACAGGATCAGAAGAACTTAGATCAT
TATATAATACAGTAGCAACCCTCTATTGTGTGCATCAAAGGATAGAGATAAAAGAC
ACCAAGGAAGCTTTAGACAAGATAGAGGAAGAGCAAAACAAAAGTAAGACCACCG
CACAGCAAGCGGCCGCTGATCTTCAGACCTGGAGGAGGAGATATGAGGGACAATTG
GAGAAGTGAATTATATAAATATAAAGTAGTAAAAATTGAACCATTAGGAGTAGCAC
CCACCAAGGCAAAGAGAAGAGTGGTGCAGAGAGAAAAAAGAGCAGTGGGAATAGG
AGCTTTGTTCCTTGGGTTCTTGGGAGCAGCAGGAAGCACTATGGGCGCAGCCTCAAT
GACGCTGACGGTACAGGCCAGACAATTATTGTCTGGTATAGTGCAGCAGCAGAACA
ATTTGCTGAGGGCTATTGAGGCGCAACAGCATCTGTTGCAACTCACAGTCTGGGGCA
TCAAGCAGCTCCAGGCAAGAATCCTGGCTGTGGAAAGATACCTAAAGGATCAACAG
CTCCTGGGGATTTGGGGTTGCTCTGGAAAACTCATTTGCACCACTGCTGTGCCTTGG
AATGCTAGTTGGAGTAATAAATCTCTGGAACAGATTGGAATCACACGACCTGGATG
GAGTGGGACAGAGAAATTAACAATTACACAAGCTTAATACACTCCTTAATTGAAGA
ATCGCAAAACCAGCAAGAAAAGAATGAACAAGAATTATTGGAATTAGATAAATGGG
CAAGTTTGTGGAATTGGTTTAACATAACAAATTGGCTGTGGTATATAAAATTATTCA
TAATGATAGTAGGAGGCTTGGTAGGTTTAAGAATAGTTTTTGCTGTACTTTCTATAGT
GAATAGAGTTAGGCAGGGATATTCACCATTATCGTTTCAGACCCACCTCCCAACCCC
GAGGGGACCCGACAGGCCCGAAGGAATAGAAGAAGAAGGTGGAGAGAGAGACAG
AGACAGATCCATTCGATTAGTGAACGGATCTCGACGGTATCGATAAGCTAATTCACA
AATGGCAGTATTCATCCACAATTTTAAAAGAAAAGGGGGGATTGGGGGGTACAGTG
CAGGGGAAAGAATAGTAGACATAATAGCAACAGACATACAAACTAAAGAATTACA
AAAACAAATTACAAAAATTCAAAATTTTCGGGTTTATTACAGGGACAGCAGAGATC
CAGTTTGGGAATTAGCTTGATCGATTAGTCCAATTTGTTAAAGACAGGATATCAGTG
GTCCAGGCTCTAGTTTTGACTCAACAATATCACCAGCTGAAGCCTATAGAGTACGAG
CCATAGATAGAATAAAAGATTTTATTTAGTCTCCAGAAAAAGGGGGGAATGAAAGA
CCCCACCTGTAGGTTTGGCAAGCTAGGATCAAGGTTAGGAACAGAGAGACAGCAGA
ATATGGGCCAAACAGGATATCTGTGGTAAGCAGTTCCTGCCCCGGCTCAGGGCCAA
GAACAGTTGGAACAGCAGAATATGGGCCAAACAGGATATCTGTGGTAAGCAGTTCC
TGCCCCGGCTCAGGGCCAAGAACAGATGGTCCCCAGATGCGGTCCCGCCCTCAGCA
GTTTCTAGAGAACCATCAGATGTTTCCAGGGTGCCCCAAGGACCTGAAATGACCCTG
TGCCTTATTTGAACTAACCAATCAGTTCGCTTCTCGCTTCTGTTCGCGCGCTTCTGCT
CCCCGAGCTCAATAAAAGAGCCCACAACCCCTCACTCGGCGCGATCTAGATCTCGA
ATCGAATTCGCCACCATGCTTTCCCTTCTCCACGCAAGTACGCTCGCCGTTTTGGGCG
CTCTTTGTGTGTATGGAGCAGGTCATCTTGAGCAACCGCAGATTTCCTCCACCAAGA
CTTTGTCCAAGACCGCGCGCTTGGAGTGCGTGGTGTCAGGAATTACCATCTCAGCGA
CCAGCGTTTACTGGTACCGCGAGCGGCCAGGAGAAGTGATACAATTCTTGGTATCAA
TAAGCTACGATGGAACAGTTCGGAAAGAATCTGGCATTCCATCCGGTAAATTTGAG
GTCGATCGGATTCCCGAAACTTCAACCTCCACGCTGACCATCCACAATGTAGAGAAG
CAGGATATTGCGACGTATTACTGTGCGCTTTGGGAAGCACAGCAGGAACTGGGCAAAA
AAATAAAAGTTTTTGGACCAGGAACAAAACTGATAATTACGGATAAACAGCTTGATGCAG
ATGTGTCCCCAAAACCTACAATTTTCTTGCCTTCCATAGCCGAGACTAAGCTCCAAA
AAGCTGGAACTTATCTTTGCCTCCTGGAGAAATTCTTTCCTGATGTGATTAAGATCCA
TTGGGAGGAGAAGAAATCAAATACGATTCTCGGCAGCCAAGAAGGCAACACCATGA
AAACGAATGATACCTACATGAAGTTTAGTTGGCTGACGGTGCCTGAGAAATCTCTGG
ACAAAGAGCACAGGTGTATTGTGAGGCACGAAAACAACAAAAATGGTGTGGACCA
GGAAATCATATTCCCCCCGATAAAGACTGATGTAATTACAATGGACCCCAAAGATA
ATTGCAGCAAAGACGCCAATGATACTTTGCTGCTTCAGCTGACCAACACTAGCGCCT
ACTATATGTACTTGCTTCTGTTGCTGAAGTCTGTCGTATACTTCGCAATCATCACATG
TTGTTTGCTCAGGAGGACCGCGTTTTGTTGCAACGGTGAGAAATCTAGAGCCAAGCG
GGGCTCTGGCGAGGGCAGAGGCTCTCTGCTGACCTGCGGAGATGTGGAAGAAAATC
CCGGCCCTATGCAAAGAATCTCATCCCTCATTCATCTCTCACTTTTTTGGGCAGGGGT
AATGTCTGCTATCGAACTTGTTCCTGAACACCAGACTGTACCGGTATCCATTGGGGT
CCCGGCAACTCTTCGGTGCTCCATGAAGGGGGAAGCCATCGGGAATTACTATATCAA
CTGGTACCGGAAAACCCAGGGTAATACCATGACTTTCATTTATAGAGAAAAGGACA
TATATGGTCCTGGCTTTAAAGACAATTTCCAGGGTGATATCGACATAGCTAAGAACC
TTGCAGTCTTGAAAATCCTGGCTCCTAGCGAACGAGATGAAGGCAGCTACTATTGTG
CGTGTGACACGCTCGGAATGGGAGGGGAATACACTGACAAACTCATCTTCGGAAAGGGTA
CCAGAGTGACAGTAGAGCCAAGGAGCCAACCGCATACAAAACCTTCTGTTTTTGTGAT
GAAGAATGGAACGAATGTTGCTTGCTTGGTCAAAGAATTTTATCCAAAAGATATAA
GAATAAATCTCGTGAGTTCAAAAAAGATTACAGAATTTGATCCCGCCATTGTGATAT
CCCCTTCCGGTAAGTATAATGCTGTAAAATTGGGTAAATATGAAGACAGCAACAGC
GTAACTTGTTCTGTCCAACATGATAATAAAACGGTTCACTCTACCGACTTTGAAGTG
AAGACTGATTCTACGGATCATGTTAAACCCAAAGAGACGGAAAATACAAAGCAGCC
GAGTAAATCATGCCATAAACCCAAGGCAATCGTTCACACAGAAAAGGTAAATATGA
TGAGCCTTACTGTCCTGGGACTGAGAATGCTTTTTGCTAAGACCGTTGCGGTGAATT
TCCTTCTTACTGCTAAGCTCTTCTTTCTCTAATGAGTTAACCTCGAGGGATCCCCCGG
GGTCGACAATCAACCTCTGGATTACAAAATTTGTGAAAGATTGACTGGTATTCTTAA
CTATGTTGCTCCTTTTACGCTATGTGGATACGCTGCTTTAATGCCTTTGTATCATGCT
ATTGCTTCCCGTATGGCTTTCATTTTCTCCTCCTTGTATAAATCCTGGTTGCTGTCTCT
TTATGAGGAGTTGTGGCCCGTTGTCAGGCAACGTGGCGTGGTGTGCACTGTGTTTGC
TGACGCAACCCCCACTGGTTGGGGCATTGCCACCACCTGTCAGCTCCTTTCCGGGAC
TTTCGCTTTCCCCCTCCCTATTGCCACGGCGGAACTCATCGCCGCCTGCCTTGCCCGC
TGCTGGACAGGGGCTCGGCTGTTGGGCACTGACAATTCCGTGGTGTTGTCGGGGAAA
TCATCGTCCTTTCCTTGGCTGCTCGCCTGTGTTGCCACCTGGATTCTGCGCGGGACGT
CCTTCTGCTACGTCCCTTCGGCCCTCAATCCAGCGGACCTTCCTTCCCGCGGCCTGCT
GCCGGCTCTGCGGCCTCTTCCGCGTCTTCGCCTTCGCCCTCAGACGAGTCGGATCTCC
CTTTGGGCCGCCTCCCCGCCTGGAATTAATTCGAGCTCGGTACCTTTAAGACCAATG
ACTTACAAGGCAGCTGTAGATCTTAGCCACTTTTTAAAAGAAAAGGGGGGACTGGA
AGGGCTAATTCACTCCCAACGAAGACAAGATCTGCTTTTTGCTTGTACTGGGTCTCT
CTGGTTAGACCAGATCTGAGCCTGGGAGCTCTCTGGCTAACTAGGGAACCCACTGCT
TAAGCCTCAATAAAGCTTGCCTTGAGTGCTTCAAGTAGTGTGTGCCCGTCTGTTGTGT
GACTCTGGTAACTAGAGATCCCTCAGACCCTTTTAGTCAGTGTGGAAAATCTCTAGC
AGTAGTAGTTCATGTCATCTTATTATTCAGTATTTATAACTTGCAAAGAAATGAATAT
CAGAGAGTGAGAGGAACTTGTTTATTGCAGCTTATAATGGTTACAAATAAAGCAAT
AGCATCACAAATTTCACAAATAAAGCATTTTTTTCACTGCATTCTAGTTGTGGTTTGT
CCAAACTCATCAATGTATCTTATCATGTCTGGCTCTAGCTATCCCGCCCCTAACTCCG
CCCATCCCGCCCCTAACTCCGCCCAGTTCCGCCCATTCTCCGCCCCATGGCTGACTA
ATTTTTTTTATTTATGCAGAGGCCGAGGCCGCCTCGGCCTCTGAGCTATTCCAGAAGT
AGTGAGGAGGCTTTTTTGGAGGCCTAGGCTTTTGCGTCGAGACGTACCCAATTCGCC
CTATAGTGAGTCGTATTACGCGCGCTCACTGGCCGTCGTTTTACAACGTCGTGACTG
GGAAAACCCTGGCGTTACCCAACTTAATCGCCTTGCAGCACATCCCCCTTTCGCCAG
CTGGCGTAATAGCGAAGAGGCCCGCACCGATCGCCCTTCCCAACAGTTGCGCAGCC
TGAATGGCGAATGGCGCGACGCGCCCTGTAGCGGCGCATTAAGCGCGGCGGGTGTG
GTGGTTACGCGCAGCGTGACCGCTACACTTGCCAGCGCCCTAGCGCCCGCTCCTTTC
GCTTTCTTCCCTTCCTTTCTCGCCACGTTCGCCGGCTTTCCCCGTCAAGCTCTAAATC
GGGGGCTCCCTTTAGGGTTCCGATTTAGTGCTTTACGGCACCTCGACCCCAAAAAAC
TTGATTAGGGTGATGGTTCACGTAGTGGGCCATCGCCCTGATAGACGGTTTTTCGCC
CTTTGACGTTGGAGTCCACGTTCTTTAATAGTGGACTCTTGTTCCAAACTGGAACAA
CACTCAACCCTATCTCGGTCTATTCTTTTGATTTATAAGGGATTTTGCCGATTTCGGC
CTATTGGTTAAAAAATGAGCTGATTTAACAAAAATTTAACGCGAATTTTAACAAAAT
ATTAACGTTTACAATTTCC
pMNDW-γδ1 DNA sequence:
TCRγ9(γδ1 CDR3)-T2A-TCRδ2(γδ1 CDR3)
(SEQ ID NO: 54)
CAGGTGGCACTTTTCGGGGAAATGTGCGCGGAACCCCTATTTGTTTATTTTTCTAAAT
ACATTCAAATATGTATCCGCTCATGAGACAATAACCCTGATAAATGCTTCAATAATA
TTGAAAAAGGAAGAGTATGAGTATTCAACATTTCCGTGTCGCCCTTATTCCCTTTTTT
GCGGCATTTTGCCTTCCTGTTTTTGCTCACCCAGAAACGCTGGTGAAAGTAAAAGAT
GCTGAAGATCAGTTGGGTGCACGAGTGGGTTACATCGAACTGGATCTCAACAGCGG
TAAGATCCTTGAGAGTTTTCGCCCCGAAGAACGTTTTCCAATGATGAGCACTTTTAA
AGTTCTGCTATGTGGCGCGGTATTATCCCGTATTGACGCCGGGCAAGAGCAACTCGG
TCGCCGCATACACTATTCTCAGAATGACTTGGTTGAGTACTCACCAGTCACAGAAAA
GCATCTTACGGATGGCATGACAGTAAGAGAATTATGCAGTGCTGCCATAACCATGA
GTGATAACACTGCGGCCAACTTACTTCTGACAACGATCGGAGGACCGAAGGAGCTA
ACCGCTTTTTTGCACAACATGGGGGATCATGTAACTCGCCTTGATCGTTGGGAACCG
GAGCTGAATGAAGCCATACCAAACGACGAGCGTGACACCACGATGCCTGTAGCAAT
GGCAACAACGTTGCGCAAACTATTAACTGGCGAACTACTTACTCTAGCTTCCCGGCA
ACAATTAATAGACTGGATGGAGGCGGATAAAGTTGCAGGACCACTTCTGCGCTCGG
CCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCT
ACACGACGGGGAGTCAGGCAACTATGGATGAACGAAATAGACAGATCGCTGAGATA
GGTGCCTCACTGATTAAGCATTGGTAACTGTCAGACCAAGTTTACTCATATATACTTT
AGATTGATTTAAAACTTCATTTTTAATTTAAAAGGATCTAGGTGAAGATCCTTTTTGA
TAATCTCATGACCAAAATCCCTTAACGTGAGTTTTCGTTCCACTGAGCGTCAGACCC
CGTAGAAAAGATCAAAGGATCTTCTTGAGATCCTTTTTTTCTGCGCGTAATCTGCTGC
TTGCAAACAAAAAAACCACCGCTACCAGCGGTGGTTTGTTTGCCGGATCAAGAGCT
ACCAACTCTTTTTCCGAAGGTAACTGGCTTCAGCAGAGCGCAGATACCAAATACTGT
CCTTCTAGTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGTCGGGCT
GAACGGGGGGTTCGTGCACACAGCCCAGCTTGGAGCGAACGACCTACACCGAACTG
AGATACCTACAGCGTGAGCTATGAGAAAGCGCCACGCTTCCCGAAGGGAGAAAGGC
GGACAGGTATCCGGTAAGCGGCAGGGTCGGAACAGGAGAGCGCACGAGGGAGCTT
CCAGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCCAG
CAACGCGGCCTTTTTACGGTTCCTGGCCTTTTGCTGGCCTTTTGCTCACATGTTCTTTC
CTGCGTTATCCCCTGATTCTGTGGATAACCGTATTACCGCCTTTGAGTGAGCTGATAC
CGCTCGCCGCAGCCGAACGACCGAGCGCAGCGAGTCAGTGAGCGAGGAAGCGGAA
GAGCGCCCAATACGCAAACCGCCTCTCCCCGCGCGTTGGCCGATTCATTAATGCAGC
TGGCACGACAGGTTTCCCGACTGGAAAGCGGGCAGTGAGCGCAACGCAATTAATGT
GAGTTAGCTCACTCATTAGGCACCCCAGGCTTTACACTTTATGCTTCCGGCTCGTATG
TTGTGTGGAATTGTGAGCGGATAACAATTTCACACAGGAAACAGCTATGACCATGAT
TACGCCAAGCGCGCAATTAACCCTCACTAAAGGGAACAAAAGCTGGAGCTGCAAGC
TTGGCCATTGCATACGTTGTATCCATATCATAATATGTACATTTATATTGGCTCATGT
CCAACATTACCGCCATGTTGACATTGATTATTGACTAGTTATTAATAGTAATCAATTA
CGGGGTCATTAGTTCATAGCCCATATATGGAGTTCCGCGTTACATAACTTACGGTAA
ATGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGAGTATT
TACGGTAAACTGCCCACTTGGCAGTACATCAAGTGTATCATATGCCAAGTACGCCCC
CTATTGACGTCAATGACGGTAAATGGCCCGCCTGGCATTATGCCCAGTACATGACCT
TATGGGACTTTCCTACTTGGCAGTACATCTACGTATTAGTCATCGCTATTACCATGGT
GATGCGGTTTTGGCAGTACATCAATGGGCGTGGATAGCGGTTTGACTCACGGGGATT
TCCAAGTCTCCACCCCATTGACGTCAATGGGAGTTTGTTTTGGCACCAAAATCAACG
GGACTTTCCAAAATGTCGTAACAACTCCGCCCCATTGACGCAAATGGGCGGTAGGC
GTGTACGGTGGGAGGTCTATATAAGCAGAGCTCGTTTAGTGAACCGGGGTCTCTCTG
GTTAGACCAGATCTGAGCCTGGGAGCTCTCTGGCTAACTAGGGAACCCACTGCTTAA
GCCTCAATAAAGCTTGCCTTGAGTGCTTCAAGTAGTGTGTGCCCGTCTGTTGTGTGA
CTCTGGTAACTAGAGATCCCTCAGACCCTTTTAGTCAGTGTGGAAAATCTCTAGCAG
TGGCGCCCGAACAGGGACCTGAAAGCGAAAGGGAAACCAGAGGAGCTCTCTCGAC
GCAGGACTCGGCTTGCTGAAGCGCGCACGGCAAGAGGCGAGGGGCGGCGACTGGT
GAGTACGCCAAAAATTTTGACTAGCGGAGGCTAGAAGGAGAGAGATGGGTGCGAG
AGCGTCAGTATTAAGCGGGGGAGAATTAGATCGCGATGGGAAAAAATTCGGTTAAG
GCCAGGGGGAAAGAAAAAATATAAATTAAAACATATAGTATGGGCAAGCAGGGAG
CTAGAACGATTCGCAGTTAATCCTGGCCTGTTAGAAACATCAGAAGGCTGTAGACA
AATACTGGGACAGCTACAACCATCCCTTCAGACAGGATCAGAAGAACTTAGATCAT
TATATAATACAGTAGCAACCCTCTATTGTGTGCATCAAAGGATAGAGATAAAAGAC
ACCAAGGAAGCTTTAGACAAGATAGAGGAAGAGCAAAACAAAAGTAAGACCACCG
CACAGCAAGCGGCCGCTGATCTTCAGACCTGGAGGAGGAGATATGAGGGACAATTG
GAGAAGTGAATTATATAAATATAAAGTAGTAAAAATTGAACCATTAGGAGTAGCAC
CCACCAAGGCAAAGAGAAGAGTGGTGCAGAGAGAAAAAAGAGCAGTGGGAATAGG
AGCTTTGTTCCTTGGGTTCTTGGGAGCAGCAGGAAGCACTATGGGCGCAGCCTCAAT
GACGCTGACGGTACAGGCCAGACAATTATTGTCTGGTATAGTGCAGCAGCAGAACA
ATTTGCTGAGGGCTATTGAGGCGCAACAGCATCTGTTGCAACTCACAGTCTGGGGCA
TCAAGCAGCTCCAGGCAAGAATCCTGGCTGTGGAAAGATACCTAAAGGATCAACAG
CTCCTGGGGATTTGGGGTTGCTCTGGAAAACTCATTTGCACCACTGCTGTGCCTTGG
AATGCTAGTTGGAGTAATAAATCTCTGGAACAGATTGGAATCACACGACCTGGATG
GAGTGGGACAGAGAAATTAACAATTACACAAGCTTAATACACTCCTTAATTGAAGA
ATCGCAAAACCAGCAAGAAAAGAATGAACAAGAATTATTGGAATTAGATAAATGGG
CAAGTTTGTGGAATTGGTTTAACATAACAAATTGGCTGTGGTATATAAAATTATTCA
TAATGATAGTAGGAGGCTTGGTAGGTTTAAGAATAGTTTTTGCTGTACTTTCTATAGT
GAATAGAGTTAGGCAGGGATATTCACCATTATCGTTTCAGACCCACCTCCCAACCCC
GAGGGGACCCGACAGGCCCGAAGGAATAGAAGAAGAAGGTGGAGAGAGAGACAG
AGACAGATCCATTCGATTAGTGAACGGATCTCGACGGTATCGATAAGCTAATTCACA
AATGGCAGTATTCATCCACAATTTTAAAAGAAAAGGGGGGATTGGGGGGTACAGTG
CAGGGGAAAGAATAGTAGACATAATAGCAACAGACATACAAACTAAAGAATTACA
AAAACAAATTACAAAAATTCAAAATTTTCGGGTTTATTACAGGGACAGCAGAGATC
CAGTTTGGGAATTAGCTTGATCGATTAGTCCAATTTGTTAAAGACAGGATATCAGTG
GTCCAGGCTCTAGTTTTGACTCAACAATATCACCAGCTGAAGCCTATAGAGTACGAG
CCATAGATAGAATAAAAGATTTTATTTAGTCTCCAGAAAAAGGGGGGAATGAAAGA
CCCCACCTGTAGGTTTGGCAAGCTAGGATCAAGGTTAGGAACAGAGAGACAGCAGA
ATATGGGCCAAACAGGATATCTGTGGTAAGCAGTTCCTGCCCCGGCTCAGGGCCAA
GAACAGTTGGAACAGCAGAATATGGGCCAAACAGGATATCTGTGGTAAGCAGTTCC
TGCCCCGGCTCAGGGCCAAGAACAGATGGTCCCCAGATGCGGTCCCGCCCTCAGCA
GTTTCTAGAGAACCATCAGATGTTTCCAGGGTGCCCCAAGGACCTGAAATGACCCTG
TGCCTTATTTGAACTAACCAATCAGTTCGCTTCTCGCTTCTGTTCGCGCGCTTCTGCT
CCCCGAGCTCAATAAAAGAGCCCACAACCCCTCACTCGGCGCGATCTAGATCTCGA
ATCGAATTCGCCACCATGCTTTCCCTTCTCCACGCAAGTACGCTCGCCGTTTTGGGCG
CTCTTTGTGTGTATGGAGCAGGTCATCTTGAGCAACCGCAGATTTCCTCCACCAAGA
CTTTGTCCAAGACCGCGCGCTTGGAGTGCGTGGTGTCAGGAATTACCATCTCAGCGA
CCAGCGTTTACTGGTACCGCGAGCGGCCAGGAGAAGTGATACAATTCTTGGTATCAA
TAAGCTACGATGGAACAGTTCGGAAAGAATCTGGCATTCCATCCGGTAAATTTGAG
GTCGATCGGATTCCCGAAACTTCAACCTCCACGTTAACCATCCACAATGTAGAGAAG
CAGGATATTGCGACGTATTACTGTGCGCTTTGGGAAGTACGCGAACTGGGCAAAAAAAT
AAAAGTTTTTGGACCAGGAACAAAACTGATAATTACGGATAAACAGCTTGATGCAGATGT
GTCCCCAAAACCTACAATTTTCTTGCCTTCCATAGCCGAGACTAAGCTCCAAAAAGC
TGGAACTTATCTTTGCCTCCTGGAGAAATTCTTTCCTGATGTGATTAAGATCCATTGG
GAGGAGAAGAAATCAAATACGATTCTCGGCAGCCAAGAAGGCAACACCATGAAAA
CGAATGATACCTACATGAAGTTTAGTTGGCTGACGGTGCCTGAGAAATCTCTGGACA
AAGAGCACAGGTGTATTGTGAGGCACGAAAACAACAAAAATGGTGTGGACCAGGA
AATCATATTCCCCCCGATAAAGACTGATGTAATTACAATGGACCCCAAAGATAATTG
CAGCAAAGACGCCAATGATACTTTGCTGCTTCAGCTGACCAACACTAGCGCCTACTA
TATGTACTTGCTTCTGTTGCTGAAGTCTGTCGTATACTTCGCAATCATCACATGTTGT
TTGCTCAGGAGGACCGCGTTTTGTTGCAACGGTGAGAAATCTAGAGCCAAGCGGGG
CTCTGGCGAGGGCAGAGGCTCTCTGCTGACCTGCGGAGATGTGGAAGAAAATCCCG
GCCCTATGCAAAGAATCTCATCCCTCATTCATCTCTCACTTTTTTGGGCAGGGGTAAT
GTCTGCTATCGAACTTGTTCCTGAACACCAGACTGTACCGGTATCCATTGGGGTCCC
GGCAACTCTTCGGTGCTCCATGAAGGGGGAAGCCATCGGGAATTACTATATCAACTG
GTACCGGAAAACCCAGGGTAATACCATGACTTTCATTTATAGAGAAAAGGACATAT
ATGGTCCTGGCTTTAAAGACAATTTCCAGGGTGATATCGACATAGCTAAGAACCTTG
CAGTCTTGAAAATCCTGGCTCCTAGCGAACGAGATGAAGGCAGCTACTATTGTGCGT
GTGACACGGTAGGGGGTGCAACTGACAAACTCATCTTCGGAAAGGGTACCAGAGTGACA
GTAGAGCCAAGGAGCCAACCGCATACAAAACCTTCTGTTTTTGTGATGAAGAATGGA
ACGAATGTTGCTTGCTTGGTCAAAGAATTTTATCCAAAAGATATAAGAATAAATCTC
GTGAGTTCAAAAAAGATTACAGAATTTGATCCCGCCATTGTGATATCCCCTTCCGGT
AAGTATAATGCTGTAAAATTGGGTAAATATGAAGACAGCAACAGCGTAACTTGTTCT
GTCCAACATGATAATAAAACGGTTCACTCTACCGACTTTGAAGTGAAGACTGATTCT
ACGGATCATGTTAAACCCAAAGAGACGGAAAATACAAAGCAGCCGAGTAAATCATG
CCATAAACCCAAGGCAATCGTTCACACAGAAAAGGTAAATATGATGAGCCTTACTG
TCCTGGGACTGAGAATGCTTTTTGCTAAGACCGTTGCGGTGAATTTCCTTCTTACTGC
TAAGCTCTTCTTTCTCTAATGAGGATCCCCCGGGGTCGACAATCAACCTCTGGATTA
CAAAATTTGTGAAAGATTGACTGGTATTCTTAACTATGTTGCTCCTTTTACGCTATGT
GGATACGCTGCTTTAATGCCTTTGTATCATGCTATTGCTTCCCGTATGGCTTTCATTTT
CTCCTCCTTGTATAAATCCTGGTTGCTGTCTCTTTATGAGGAGTTGTGGCCCGTTGTC
AGGCAACGTGGCGTGGTGTGCACTGTGTTTGCTGACGCAACCCCCACTGGTTGGGGC
ATTGCCACCACCTGTCAGCTCCTTTCCGGGACTTTCGCTTTCCCCCTCCCTATTGCCA
CGGCGGAACTCATCGCCGCCTGCCTTGCCCGCTGCTGGACAGGGGCTCGGCTGTTGG
GCACTGACAATTCCGTGGTGTTGTCGGGGAAATCATCGTCCTTTCCTTGGCTGCTCGC
CTGTGTTGCCACCTGGATTCTGCGCGGGACGTCCTTCTGCTACGTCCCTTCGGCCCTC
AATCCAGCGGACCTTCCTTCCCGCGGCCTGCTGCCGGCTCTGCGGCCTCTTCCGCGT
CTTCGCCTTCGCCCTCAGACGAGTCGGATCTCCCTTTGGGCCGCCTCCCCGCCTGGA
ATTAATTCGAGCTCGGTACCTTTAAGACCAATGACTTACAAGGCAGCTGTAGATCTT
AGCCACTTTTTAAAAGAAAAGGGGGGACTGGAAGGGCTAATTCACTCCCAACGAAG
ACAAGATCTGCTTTTTGCTTGTACTGGGTCTCTCTGGTTAGACCAGATCTGAGCCTGG
GAGCTCTCTGGCTAACTAGGGAACCCACTGCTTAAGCCTCAATAAAGCTTGCCTTGA
GTGCTTCAAGTAGTGTGTGCCCGTCTGTTGTGTGACTCTGGTAACTAGAGATCCCTC
AGACCCTTTTAGTCAGTGTGGAAAATCTCTAGCAGTAGTAGTTCATGTCATCTTATTA
TTCAGTATTTATAACTTGCAAAGAAATGAATATCAGAGAGTGAGAGGAACTTGTTTA
TTGCAGCTTATAATGGTTACAAATAAAGCAATAGCATCACAAATTTCACAAATAAAG
CATTTTTTTCACTGCATTCTAGTTGTGGTTTGTCCAAACTCATCAATGTATCTTATCAT
GTCTGGCTCTAGCTATCCCGCCCCTAACTCCGCCCATCCCGCCCCTAACTCCGCCCA
GTTCCGCCCATTCTCCGCCCCATGGCTGACTAATTTTTTTTATTTATGCAGAGGCCGA
GGCCGCCTCGGCCTCTGAGCTATTCCAGAAGTAGTGAGGAGGCTTTTTTGGAGGCCT
AGGCTTTTGCGTCGAGACGTACCCAATTCGCCCTATAGTGAGTCGTATTACGCGCGC
TCACTGGCCGTCGTTTTACAACGTCGTGACTGGGAAAACCCTGGCGTTACCCAACTT
AATCGCCTTGCAGCACATCCCCCTTTCGCCAGCTGGCGTAATAGCGAAGAGGCCCGC
ACCGATCGCCCTTCCCAACAGTTGCGCAGCCTGAATGGCGAATGGCGCGACGCGCC
CTGTAGCGGCGCATTAAGCGCGGCGGGTGTGGTGGTTACGCGCAGCGTGACCGCTA
CACTTGCCAGCGCCCTAGCGCCCGCTCCTTTCGCTTTCTTCCCTTCCTTTCTCGCCAC
GTTCGCCGGCTTTCCCCGTCAAGCTCTAAATCGGGGGCTCCCTTTAGGGTTCCGATTT
AGTGCTTTACGGCACCTCGACCCCAAAAAACTTGATTAGGGTGATGGTTCACGTAGT
GGGCCATCGCCCTGATAGACGGTTTTTCGCCCTTTGACGTTGGAGTCCACGTTCTTTA
ATAGTGGACTCTTGTTCCAAACTGGAACAACACTCAACCCTATCTCGGTCTATTCTTT
TGATTTATAAGGGATTTTGCCGATTTCGGCCTATTGGTTAAAAAATGAGCTGATTTA
ACAAAAATTTAACGCGAATTTTAACAAAATATTAACGTTTACAATTTCC

All publications mentioned herein (e.g., PCT Published International Application Nos. PCT/US19/36786 and. PCT/US2020/037486; U.S. patent application Ser. No. 15/320,037; as well as Zarin et al., Cell Immunol. 2015 July; 296(1):70-5. doi: 10.1016/j.cellimm.2015.03.007. Epub 2015, those listed above etc.) are incorporated by reference to disclose and describe aspects, methods and/or materials in connection with the cited publications. Many of the techniques and procedures described or referenced herein are well understood and commonly employed by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

1. An engineered cell which is a cell genetically modified to contain at least one exogenous gamma delta T cell receptor (γδ TCR) nucleic acid molecule.

2. The engineered cell of claim 1, wherein the cell is a pluripotent stem cell, a hematopoietic stem cell, a hematopoietic progenitor cell, or an immune cell.

3. The engineered cell of claim 1, wherein the cell is a human cell.

4. The engineered cell of claim 1, wherein the γδ TCR nucleic acid molecule is a clone of a T cell receptor of a γδ T cell or has a sequence that has been modified from that of the T cell receptor of the γδ T cell.

5. The engineered cell of claim 1, wherein the γδ TCR nucleic acid molecule is a clone of a T cell receptor of a human γδ T cell or has a sequence that has been modified from that of the T cell receptor of the human γδ T cell.

6. The engineered cell of claim 1, wherein the γδ TCR nucleic acid molecule comprises a nucleic acid sequence obtained from a human γδ T cell receptor.

7. The engineered cell of claim 1, wherein the engineered cell lacks exogenous oncogenes.

8. The engineered cell of claim 1, wherein the gamma delta T cell receptor nucleic acid molecule encodes at least one amino acid sequence shown in SEQ ID NO: 1-SEQ ID NO: 52

9. A composition of matter comprising an engineered cell transduced with at least one polynucleotide encoding a T cell receptor gamma chain polypeptide and/or a T cell receptor delta chain polypeptide, wherein the T cell receptor gamma chain polypeptide and/or the T cell receptor delta chain polypeptide comprises at least one amino acid sequence shown in SEQ ID NO: 1-SEQ ID NO: 52.

10. A method of making an engineered functional gamma delta T cell comprising at least one exogenous nucleic acid molecule encoding a T cell receptor gamma chain polypeptide and/or a T cell receptor delta chain polypeptide, the method comprising:
transducing a human hematopoietic stem/progenitor cell with at least one exogenous nucleic acid molecule encoding the T cell receptor gamma chain polypeptide and the T cell receptor delta chain polypeptide so that the human pluripotent cell transduced by the at least one exogenous nucleic acid molecule expresses a T cell receptor comprising a gamma chain polypeptide and a delta chain polypeptide encoded by the at least one exogenous nucleic acid molecule; and
differentiating the human hematopoietic stem/progenitor cell so as to generate the engineered functional gamma delta T cell.

11. The method of claim 10, wherein the method comprises:
(a) differentiating transduced human hematopoietic stem/progenitor cells in a first in vitro culture; and further
(b) expanding the differentiated cells of (a) in a second in vitro culture.

12. The method of claim 11, wherein:
the hematopoietic stem/progenitor cells are cultured in a medium that does not comprise feeder cells; and/or
the hematopoietic stem/progenitor cells are cultured in a medium comprising one or more of IL-3, IL-7, IL-6, SCF, MCP-4, EPO, TPO, FLT3L, and/or retronectin.

13. The method of claim 12, which further comprises expanding the cell transduced with the nucleic acid molecule encoding a T cell receptor gamma chain polypeptide and/or a T cell receptor delta chain polypeptide in vitro.

14. The method of claim 10, further comprising engrafting the hematopoietic stem/progenitor cell transduced with the nucleic acid molecule encoding the T cell receptor gamma chain polypeptide and/or the T cell receptor delta chain polypeptide in a subject so as to generate clonal populations of the engineered cell in vivo.

15. The method of claim 10, wherein the engineered functional gamma delta T cell comprises a gene expression profile characterized as being at least one of:
HLA-I-low/negative;
HLA-II-low/negative;
HLA-E-positive; and
expressing immune regulatory gene(s) and/or a suicide gene.

16. The method of claim 10, wherein:
the exogenous nucleic acid molecule is contained in a lentiviral expression vector; and/or
the method further comprises contacting the transduced cell with an agent selected to facilitate growth and/or differentiation.

17. The method of claim 16, wherein the method further comprises co-culturing the transduced cells with peripheral blood mononuclear cells, antigen presenting cells, or artificial antigen presenting cells.

18. The method of claim 10, wherein the hematopoietic stem/progenitor cell comprises a CD34⁺ hematopoietic stem or progenitor cell.

19. The method of claim 10, wherein the T cell receptor gamma chain polypeptide and/or the T cell receptor delta chain polypeptide comprises at least one amino acid sequence shown in SEQ ID NO: 1-SEQ ID NO: 52.

20. An engineered functional gamma delta T cell produced by the method of any one of claims 10-19.

21. A method of treating a subject in need of gamma delta T cells, which comprises administering to the subject a cell of claims 1-9 or 20.

22. The method of claim 21, wherein the gamma delta T cells are generated by transducing a CD34⁺ hematopoietic stem or progenitor cell with at least one exogenous nucleic acid molecule encoding a T cell receptor gamma chain polypeptide, a T cell receptor delta chain polypeptide, IL-15, and a suicide gene.

23. The method of claim 22, wherein the gamma delta T cell comprises at least one amino acid sequence shown in SEQ ID NO: 1-SEQ ID NO: 52.

24. The method of claim 21, wherein:
the subject in need of gamma delta T cells is diagnosed with a cancer; or
the subject in need of gamma delta T cells is diagnosed with a viral, fungal or protozoal infection.

25. The method of claim 21, wherein the T cell receptor gamma chain polypeptide and T cell receptor delta chain polypeptide are selected from a γδ T cell receptor observed to target cancer cells or cells infected with a virus, fungi or protozoan.
